(19) United States
(12) Patent Application Publication
(10) Pub. No.: US 2024/0058753 A1
(43) Pub. Date: Feb. 22, 2024

(54) ELECTROPHORETIC DEVICES AND METHODS FOR NEXT-GENERATION SEQUENCING LIBRARY PREPARATION

(71) Applicants: Roche Sequencing Solutions, Inc., Pleasanton, CA (US); Kapa Biosystems, Inc., Wilmington, MA (US)

(72) Inventors: Yann Astier, Livermore, CA (US); Taariq Firfirey, Cape Town (ZA); Jaeyoung Yang, Pleasanton, CA (US)

(21) Appl. No.: 18/031,087

(22) PCT Filed: Oct. 13, 2021
(86) PCT No.: PCT/US2021/054826

§ 371 Date: Apr. 10, 2023

Related U.S. Application Data
(60) Provisional application No. 63/092,318, filed on Oct. 15, 2020.

(51) Int. Cl. B01D 57/02; C12Q 1/6806

(52) U.S. Cl. CPC B01D 57/02; C12Q 1/6806

(57) ABSTRACT

The present disclosure is directed to automated systems including an electrophoretic device including one or more separation conduits. In some embodiments, the automated systems are suitable for use in sample cleanup and/or target enrichment processes, such as sample cleanup and/or target enrichment processes conducted prior to sequencing, e.g., next generation sequencing.

### CROSS-REFERENCE TO RELATED APPLICATIONS

The present application is a National Stage of International Application No. PCT/US2021/054826 filed on Oct. 13, 2021, which application claims the benefit of the filing date of U.S. provisional application No. 63/092,318 filed on Oct. 15, 2020, the disclosure of which is hereby incorporated by reference herein in its entirety.

### FIELD OF THE DISCLOSURE

The present disclosure is directed to systems including an electrophoretic device adapted to facilitate the purification and/or enrichment of a mixture of molecules, such as nucleic acid molecules. In some embodiments, the systems are suitable for use in sample cleanup and/or target enrichment processes, such as sample cleanup and/or target enrichment processes conducted prior to sequencing a sample using next-generation sequencing.

### BACKGROUND OF THE DISCLOSURE

A number of protocols exist for particle separation. For example, methods based on magnetic control utilize surface-functionalized magnetic beads to capture target particles through specific binding and, subsequently, to separate the target particles by magnetic manipulation. This separation scheme relies on the interaction of chemical bonds rather than geometrical or physical properties of the particles and hence allows highly specific and selective particle separation.

Magnetic separation processes have been utilized for target enrichment. These magnetic separation processes often require certain steps to be repeated multiple times. For instance, the following steps are often performed during a magnetic separation process: (i) sample-bead incubation for attachment of molecules of interest onto the bead surface; (ii) bead pull-down using a magnet; (iii) removal of supernatant for eliminating non-bound molecules; and (iv) resuspension of beads with a wash buffer to remove impurities and/or weakly-bound molecules. Each of steps (ii)-(iv) may be repeated for improved specificity. Following these steps, the molecules bound to the bead surface are released into an elution buffer. This manual processing, however, is labor-intensive and requires long incubation times. In addition, such sample processing requires multiple liquid pipetting steps, which could result in bead and sample loss, not to mention sample contamination.

### BRIEF SUMMARY OF THE DISCLOSURE

Applicant has surprisingly discovered that electrophoretic separation techniques may be used to separate a subset of molecules from a mixture of molecules into one or more chambers of an electrophoretic device without the need for multiple liquid pipetting steps. In this regard, Applicant has developed an electrophoretic device and method which facilitates the separation and/or purification of a subset of molecules (e.g., target nucleic acid molecules) from a mixture of molecules (e.g., a mixture including target nucleic acid molecules and non-target nucleic acid molecules). In some embodiments, the electrophoretic device and method utilize the principle of electrophoresis or isotachophoresis.

A first aspect of the present disclosure is an electrophoretic device comprising: one or more independently operable separation conduits; wherein each of the one or more independently operable separation conduits comprise at least one sample loading chamber, at least one waste collection chamber, and at least one sample collection chamber, wherein the at least one sample loading, the at least one waste collection, and the at least one sample collection chambers are in fluidic communication with each other through a branched transfer channel. In some embodiments, the at least one sample loading chamber is in communication with at least one first electrode, the at least one waste collection chamber is in communication with at least one second electrode, and the at least one sample collection chamber is in communication with at least one third electrode.

In some embodiments, the electrophoretic device further comprises at least two electrical traces or wires. In some embodiments, a first of the at least two electrical traces or wires communicatively couples the at least one first electrode to the at least one second electrode. In some embodiments, a second of the at least two electrical traces or wires communicatively couples the at least one first electrode to the at least one third electrode. In some embodiments, the at least one second and the at least one third electrodes are not communicatively coupled to one another.

In some embodiments, the electrophoretic device further comprises a control system. In some embodiments, the electrophoretic device further comprises one or more feedback control devices. In some embodiments, the electrophoretic device further comprises one or more heating and/or cooling modules.

In some embodiments, a first portion of a wall of each of the at least one sample loading chambers comprise a first aperture in communication with an inlet of the branched transfer channel. In some embodiments, the sample loading chamber includes a plurality of beads. In some embodiments, the first aperture is smaller than an average diameter of the plurality of beads within the sample loading chamber. In some embodiments, a second portion of the wall of each of the at least one sample loading chambers comprises a ductal opening. In some embodiments, the ductal opening is larger than the average diameter of the plurality of beads within the sample loading chamber.

In some embodiments, the electrophoretic device comprises no mechanically moving parts. In some embodiments, the at least one sample loading chamber comprises a volume ranging from between about 0.1 μL to about 5 mL. In some embodiments, the volume of the at least one sample loading chamber ranges from between about 0.1 mL to about 1 mL.

In some embodiments, the electrophoretic device further comprises two sample collection chambers, wherein a first of the two sample collection chambers is in communication with the branched transfer conduit; and wherein the first of the two sample collection chambers is in further communication with a second of the two sample collection chambers through an intermediate channel.

In some embodiments, the branched transfer channel is pre-loaded with a gel. In some embodiments, the gel is selected from the group consisting of agarose and PAGE.

In some embodiments, the at least one waste collection chamber and the at least one sample collection chamber are pre-loaded with one or more electrolytes. In some embodiments, the one or more electrolytes are leading electrolytes. In some embodiments, the one or more leading electrolytes are selected from HCl-Histidine, Tris/Acetate/EDTA (TAE), and Tris/Borate/EDTA (TBE). In some embodiments, the sample loading chamber includes one or more trailing electrolytes. In some embodiments, the one or more trailing electrolytes are selected from TAPS-TRIS, Tris/Acetate/EDTA (TAE), and Tris/Borate/EDTA (TBE).

A second aspect of the present disclosure is a use of an electrophoretic device for preparing a target enriched sample, wherein the electrophoretic device comprises one or more independently operable separation conduits; wherein each of the one or more independently operable separation conduits comprise at least one sample loading chamber, at least one waste collection chamber, and at least one sample collection chamber, wherein the sample loading, waste collection, and sample collection chambers are coupled to each other through a branched transfer channel. In some embodiments, the at least one sample loading chamber is in communication with at least one first electrode, the at least one waste collection chamber is in communication with at least one second electrode, and the at least one sample collection chamber is in communication with at least one third electrode.

A third aspect of the present disclosure is a system comprising an electrophoretic device and a sequencing device, wherein the electrophoretic device comprises one or more independently operable separation conduits; wherein each of the one or more independently operable separation conduits comprise at least one sample loading chamber, at least one waste collection chamber, and at least one sample collection chamber, wherein the sample loading, waste collection, and sample collection chambers are coupled to each other through a branched transfer channel. In some embodiments, the at least one sample loading chamber is in communication with at least one first electrode, the at least one waste collection chamber is in communication with at least one second electrode, and the at least one sample collection chamber is in communication with at least one third electrode. In some embodiments, the sequencing device is a next generation sequencing device.

A fourth aspect of the present disclosure is a method of obtaining a population of target nucleic acid sequences comprising: (a) introducing a pool of oligonucleotide probes to an obtained genomic sample to form target-probe complexes, wherein the pool of oligonucleotide probes comprise reference nucleic acid sequences capable of hybridizing to complementary target nucleic acid sequences within the obtained genomic sample and wherein the oligonucleotide probes comprise a first member of a pair of specific binding entities; (b) introducing a solution including the formed target-probe complexes to a sample loading chamber of a separation conduit of an electrophoretic device pre-loaded with a plurality of beads, wherein the plurality of beads are functionalized with a second member of the pair of specific binding entities to form bead-bound target-probe complexes; (c) transferring off-target nucleic acids to a waste collection chamber in communication with the sample loading chamber by establishing an electrical field between the sample loading chamber and the waste collection chamber; and (d) transferring the target nucleic acids to a sample collection chamber in communication with the sample loading chamber by establishing an electrical field between the sample loading chamber and the sample collection chamber. In some embodiments, the first member of the pair of specific binding entities is biotin. In some embodiments, the second member of the pair of specific binding entities is streptavidin.

In some embodiments, the method further comprises releasing the target-probe complexes from the plurality of beads. In some embodiments, the formed target-probe complexes comprise a cleavable moiety. In some embodiments, the cleavable moiety is cleaved with an enzyme. In some embodiments, the enzyme is an Uracil-Specific Excision Reagent enzyme.

In some embodiments, the method further comprises releasing the target nucleic acids from the target-probe complexes. In some embodiments, the target nucleic acids are released by heating at least the sample loading chamber.

In some embodiments, both the waste collection chamber and the sample collection chamber include one or more electrolytes. In some embodiments, the one or more electrolytes are leading electrolytes. In some embodiments, the leading electrolytes are selected from HCl-Histidine, Tris/Acetate/EDTA (TAE), and Tris/Borate/EDTA (TBE). In some embodiments, the sample loading chamber comprises one or more trailing electrolytes. In some embodiments, the one or more trailing electrolytes are selected from TAPS-TRIS, Tris/Acetate/EDTA (TAE), and Tris/Borate/EDTA (TBE).

In some embodiments, the method further comprises sequencing the population of target nucleic acid sequences. In some embodiments, the obtained genomic sample is a blood sample, or a blood plasma sample obtained from a mammalian subject. In some embodiments, the obtained genomic sample comprises cell-free nucleic acids. In some embodiments, the cell-free nucleic acids comprise DNA or RNA. In some embodiments, the cell-free nucleic acids have an average size ranging from between about 150 bp to about 180 bp. In some embodiments, the obtained genomic sample comprises circulating tumor DNA (ctDNA) or fetal cell-free DNA.

A fifth aspect of the present disclosure is a method of obtaining a population of target nucleic acid sequences comprising: (a) obtaining a sample comprising a first subset of functionalized molecules including a first reactive moiety; (b) forming bead-bound molecules by introducing the obtained sample to a sample loading chamber of an electrophoretic device pre-loaded with a plurality of beads, where each of the plurality of beads comprise a second reactive moiety capable of reacting with the first reactive moiety; (c) transferring non-functionalized molecules and/or impurities from the sample loading chamber to a waste collection chamber through a transfer channel pre-loaded with a gel by establishing an electrical field (e.g., an electrical potential) between the sample loading chamber and the waste collection chamber; (d) releasing the bead-bound molecules from the beads; and (e) transferring the released molecules from the sample loading chamber to a sample collection chamber through the transfer channel pre-loaded with the gel by establishing an electrical field between the sample loading chamber and the sample collection chamber.

In some embodiments, the first reactive moiety is selected from the group consisting of biotin, an antigenic molecule, an enzyme substrate, a receptor ligand, a polysaccharide, a thiolated molecule, and an amine-terminated molecule. In some embodiments, the second reactive moiety is selected from the group consisting of streptavidin, an antibody, an enzyme, a receptor, a lectin, a gold particle, and an NHS-activated moiety. In some embodiments, the first reactive moiety is biotin and the second reactive moiety is streptavidin.

In some embodiments, both the waste collection chamber and the sample collection chamber include one or more electrolytes. In some embodiments, the one or more electrolytes are leading electrolytes. In some embodiments, the leading electrolytes are selected from HCl-Histidine, Tris/Acetate/EDTA (TAE), and Tris/Borate/EDTA (TBE). In some embodiments, sample loading chamber comprises one or more trailing electrolytes. In some embodiments, the one or more trailing electrolytes are selected from TAPS-TRIS, Tris/Acetate/EDTA (TAE), and Tris/Borate/EDTA (TBE). In some embodiments, the method further comprises sequencing the population of target nucleic acid sequences.

A sixth aspect of the present disclosure is a kit comprising an electrophoretic device and one or more electrolytes, wherein the electrophoretic device comprises one or more independently operable separation conduits; wherein each of the one or more independently operable separation conduits comprise at least one sample loading chamber, at least one waste collection chamber, and at least one sample collection chamber, wherein the sample loading, waste collection, and sample collection chambers are coupled to each other through a branched transfer channel. In some embodiments, the at least one sample loading chamber is in communication with at least one first electrode, the at least one waste collection chamber is in communication with at least one second electrode, and the at least one sample collection chamber is in communication with at least one third electrode.

In some embodiments, the one or more electrolytes are leading electrolytes. In some embodiments, the leading electrolytes are selected from HCl-Histidine, Tris/Acetate/EDTA (TAE), and Tris/Borate/EDTA (TBE). In some embodiments, the one or more electrolytes are trailing electrolytes. In some embodiments, the one or more trailing electrolytes are selected from TAPS-TRIS, Tris/Acetate/EDTA (TAE), and Tris/Borate/EDTA (TBE).

A seventh aspect of the present disclosure is an electrophoretic device comprising: one or more independently operable separation conduits; wherein each of the one or more independently operable separation conduits comprise at least one sample loading chamber, at least one waste collection chamber, and at least one sample collection chamber, wherein the at least one sample loading, the at least one waste collection, and the at least one sample collection chambers are fluidically coupled to each other through a branched transfer channel; and wherein the electrophoretic device further comprises at least two electrodes, wherein a first of the at least two electrodes is in communication with the at least one waste collection chamber, and wherein a second of the at least two electrodes is in communication with the at least one sample collection chamber. In some embodiments, the electrophoretic device comprises at least three electrodes.

An eighth aspect of the present disclosure is a use of an electrophoretic device for preparing a target enriched sample, wherein the electrophoretic device comprises one or more independently operable separation conduits; wherein each of the one or more independently operable separation conduits comprise at least one sample loading chamber, at least one waste collection chamber, and at least one sample collection chamber, wherein the at least one sample loading, the at least one waste collection, and the at least one sample collection chambers are fluidically coupled to each other through a branched transfer channel; and wherein the electrophoretic device further comprises at least two electrodes, wherein a first of the at least two electrodes is in communication with the at least one waste collection chamber, and wherein a second of the at least two electrodes is in communication with the at least one sample collection chamber.

A ninth aspect of the present disclosure is an electrophoretic device comprising: one or more independently operable separation conduits; wherein each of the one or more independently operable separation conduits comprise at least one sample loading chamber, at least one waste collection chamber, and at least one sample collection chamber, and wherein the at least one sample loading, the at least one waste collection, and the at least one sample collection chambers are fluidically coupled to each other through a branched transfer channel; wherein the at least one sample loading chamber and the least one sample collection chamber are communicatively coupled to each other such that a first electric field may be established between the at least one sample loading chamber and the least one sample collection chamber; and wherein the at least one sample loading chamber and the least one waste collection chamber are communicatively coupled to each other such that a second electric field may be established between the at least one sample loading chamber and the least one waste collection chamber. In some embodiments, the at least one sample collection chamber is preloaded with a first electrolyte and wherein the at least one waste collection chamber is preloaded with a second electrolyte. In some embodiments, the at least one sample loading chamber is preloaded with a plurality of beads. In some embodiments, the plurality of beads are magnetic beads. In some embodiments, the plurality of beads are non-magnetic beads. In some embodiments, the plurality of beads are functionalized with biotin. In some embodiments, electrophoretic device is communicatively coupled to a control system.

A tenth aspect of the present disclosure is a use of an electrophoretic device for preparing a target enriched sample, wherein the electrophoretic device comprises one or more independently operable separation conduits; wherein each of the one or more independently operable separation conduits comprise at least one sample loading chamber, at least one waste collection chamber, and at least one sample collection chamber, wherein the at least one sample loading, the at least one waste collection, and the at least one sample collection chambers are fluidically coupled to each other through a branched transfer channel; wherein the at least one sample loading chamber and the least one sample collection chamber are communicatively coupled to each other such that a first electric field may be established between the at least one sample loading chamber and the least one sample collection chamber; and wherein the at least one sample loading chamber and the least one waste collection chamber are communicatively coupled to each other such that a second electric field may be established between the at least one sample loading chamber and the least one waste collection chamber.

### BRIEF DESCRIPTION OF THE FIGURES

For a general understanding of the features of the disclosure, reference is made to the drawings. In the drawings, like reference numerals have been used throughout to identify identical elements.

FIG. 1A depicts an electrophoretic device including an assembly having at least one separation conduit, a fluidics module, and a control system in accordance with one embodiment of the present disclosure.

FIG. 1B depicts an electrophoretic device including an assembly having at least three independently operable separation conduits, a fluidics module, and a control system in accordance with one embodiment of the present disclosure.

FIG. 1C depicts an electrophoretic device including an assembly having at least six independently operable separation conduits, a fluidics module, and a control system in accordance with one embodiment of the present disclosure.

FIG. 1D illustrates an assembly of an electrophoretic device comprised of multiple layers stacked together in accordance with one embodiment of the present disclosure.

FIG. 1E illustrates an assembly of an electrophoretic device, where the assembly includes a single separation conduit in accordance with one embodiment of the present disclosure.

FIG. 1F illustrates an assembly of an electrophoretic device, where the assembly includes a single separation conduit, and wherein the assembly is in communication with a thermal regulation module and/or one or more sensors in accordance with one embodiment of the present disclosure.

FIG. 1G illustrates an assembly of an electrophoretic device, where the assembly includes a single separation conduit, and wherein the assembly is in communication with heating and cooling modules and/or one or more sensors in accordance with one embodiment of the present disclosure.

FIG. 1H illustrates an electrophoretic device including an assembly having at least one separation conduit, a fluidics module, a downstream processing module, a feedback control device, and a control system in accordance with one embodiment of the present disclosure.

FIG. 2A depicts a separation conduit including three interconnected chambers in accordance with one embodiment of the present disclosure.

FIG. 2B depicts a separation conduit including three interconnected chambers in accordance with one embodiment of the present disclosure.

FIG. 2C depicts two independently operable separation conduits, each including three interconnected chambers in accordance with one embodiment of the present disclosure.

FIG. 2D depicts a separation conduit including three interconnected chambers, and where a loading channel of the separation conduit is indirectly in communication with a sample loading chamber via one or more inlet channels in accordance with one embodiment of the present disclosure.

FIG. 2E depicts a separation conduit including four interconnected chambers in accordance with one embodiment of the present disclosure.

FIG. 2F depicts a separation conduit including a sample loading chamber, a waste collection chamber, an intermediate chamber, and a sample collection chamber in accordance with one embodiment of the present disclosure.

FIG. 2G depicts a separation conduit including a sample loading chamber, a waste collection chamber, an intermediate chamber, and two sample collection chambers in accordance with one embodiment of the present disclosure.

FIG. 2H depicts a separation conduit including a sample loading chamber, a waste collection chamber, an intermediate chamber, and two sample collection chambers in accordance with one embodiment of the present disclosure.

FIG. 2I depicts a separation conduit including three interconnected chambers, and where a filter or mesh is provided between a sample loading chamber and a loading channel in accordance with one embodiment of the present disclosure.

FIG. 2J depicts a separation conduit including three interconnected chambers, and wherein each branch of a branched conduit fluidically coupling the three interconnected chambers includes or is in communication with a conductivity detector in accordance with one embodiment of the present disclosure.

FIG. 3A depicts a cut-away view of a wall of a sample loading chamber in accordance with one embodiment of the present disclosure.

FIG. 3B depicts a cut-away view of a sample loading chamber in communication with a loading channel in accordance with one embodiment of the present disclosure.

FIG. 3C depicts a cut-away view of a loading channel in communication with a sample loading chamber and one of a waste collection chamber or a sample collection chamber in accordance with one embodiment of the present disclosure.

FIG. 3D depicts a separation conduit including three interconnected chambers where a sample loading chamber includes one or more ductal openings in accordance with one embodiment of the present disclosure.

FIG. 4 illustrates the placement of electrodes and wires or traces coupling the various electrodes in accordance with one embodiment of the present disclosure.

FIG. 5 provides a flowchart illustrating the general method of separating and/or purifying molecules with an electrophoretic device in accordance with one embodiment of the present disclosure.

FIG. 6 provides a flowchart illustrating the general method of separating and/or purifying target nucleic acid molecules with an electrophoretic device in accordance with one embodiment of the present disclosure.

FIG. 7A illustrates an electrophoretic device including a branched channel, and where a sample loading chamber includes a plurality of beads in accordance with one embodiment of the present disclosure.

FIG. 7B illustrates an electrophoretic device including a branched channel, and where a sample loading chamber includes a plurality of beads in accordance with one embodiment of the present disclosure; where the sample loading chamber is communicatively coupled to the waste well; and where the sample loading chamber is communicatively coupled to the sample collection well.

FIG. 8 illustrates methods of separating and/or purifying target nucleic acid molecules with an electrophoretic device in accordance with one embodiment of the present disclosure.

FIG. 9 illustrates the transfer of molecules from a sample loading chamber to a waste collection chamber in accordance with one embodiment of the present disclosure.

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g., “one or the other but not both”) when preceded by terms of exclusivity, such as “either,” “one of” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins.

As used herein, the terms “biological sample” or “biological specimen” refer to a specimen or culture (e.g., microbiological cultures) that includes nucleic acids and/or a target nucleic acid. In some embodiments, a “biological sample” may include, but is not limited, to whole blood, serum, plasma, umbilical cord blood, chorionic villi, amniotic fluid, cerebrospinal fluid, spinal fluid, lavage fluid (e.g., bronchioalveolar, gastric, peritoneal, ductal, ear, arthroscopic), biopsy sample, urine, feces, sputum, saliva, nasal mucous, prostate fluid, semen, lymphatic fluid, bile, tears, sweat, breast milk, breast fluid, embryonic cells and fetal cells. The biological sample may be blood and may be plasma. As used herein, the term “blood” encompasses whole blood or any fractions of blood, such as serum and plasma as conventionally defined. Blood plasma refers to the fraction of whole blood resulting from centrifugation of blood treated with anticoagulants. Blood serum refers to the watery portion of fluid remaining after a blood sample has coagulated.

As used herein, the term “channel” refers to an enclosed passage through which a fluid can flow. The channel can have one or more openings for introductions removal of a fluid. Each channel may include a coating, e.g., a hydrophilic or hydrophobic coating.

As used herein, the term “conjugate” refers to two or more molecules (and/or materials such as nanoparticles) that are covalently linked into a larger construct. In some embodiments, a conjugate includes one or more biomolecules (such as peptides, proteins, enzymes, sugars, polysaccharides, lipids, glycoproteins, and lipoproteins) covalently linked to one or more other molecules, such as one or more other biomolecules.

As used herein, the term “electrophoresis” refers to the separating of electrically charged particles in a sample based on mobility of the particles relative to a fluid under the influence of an electric field. In some embodiments, it is believed that different charged particles can migrate at different speeds (commonly referred to as electrophoretic mobility) relative to a fluid in an electric field. The charged particles may have different charge polarity, charge state, particle size, and/or other characteristics. As a result, the charged particles separate from one another during migration in the fluid (e.g., a solvent or buffer solution). The separated charged particles may then be collected and further analyzed for identification and/or abundance.

As used herein, the term “enrichment” refers to the process of increasing the relative abundance of a population of molecules, e.g., nucleic acid molecules, in a sample relative to the total amount of the molecules initially present in the sample before treatment. Thus, an enrichment step provides a percentage or fractional increase rather than directly increasing for example, the copy number of the nucleic acid sequences of interest as amplification methods, such as a polymerase chain reaction, would.

As used herein, the term “fluid” refers to any liquid or liquid composition, including water, solvents, buffers, solutions (e.g., polar solvents, non-polar solvents), and/or mixtures. The fluid may be aqueous or non-aqueous. Non-liming examples of buffers include citric acid, potassium dihydrogen phosphate, boric acid, diethyl barbituric acid, piperazine-N,N′-bis(2-ethanesulfonic acid), dimethylarsinic acid, 2-(N-morpholino)ethanesulfonic acid, tris(hydroxymethyl)methylamine (TRIS), 2-(N-morpholino)ethanesulfonic acid (TAPS), N,N-bis(2-hydroxyethyl)glycine(Bicine), N-tris(hydroxymethyl)methylglycine (Tricine), 4-2-hydroxyethyl-1-piperazineethane sulfonic acid (HEPES), 2-{[tris(hydroxymethyl)methyl]amino}ethanesulfonic acid (TES), and combinations thereof. In other embodiments, the buffer may be comprised of tris(hydroxymethyl)methylamine (TRIS), 2-(N-morpholino)ethanesulfonic acid (TAPS), N,N-bis(2-hydroxyethyl)glycine(Bicine), N-tris(hydroxymethyl)methylglycine (Tricine), 4-2-hydroxyethyl-1-piperazineethanesulfonic acid (HEPES), 2-{[tris(hydroxymethyl)methyl]amino}ethanesulfonic acid (TES), or a combination thereof.

As used herein, the term “isotachophoresis” generally refers to the separation of charged particles by using an electric field to create boundaries or interfaces between materials (e.g., between the charged particles and other materials in a solution). Isotachophoresis generally uses multiple electrolytes, where the electrophoretic mobilities of sample ions are less than that of a leading electrolyte (LE) and greater than that of a trailing electrolyte (TE) that are placed in a device for isotachophoresis. In some embodiments, the leading electrolyte (LE) generally contains a relatively high mobility ion, and a trailing electrolyte (TE) generally contains a relatively low mobility ion. In some embodiments, the TE and LE ions are chosen to have effective mobilities respectively lower and higher than target analyte ions of interest. That is, the effective mobility of analyte ions is higher than that of the TE and lower than that of the LE. These target analytes have the same sign of charge as the LE and TE ions (i.e., a co-ion). An applied electric field causes LE ions to move away from TE ions and TE ions to trail behind. A moving interface forms between the adjacent and contiguous TE and LE zones. In some embodiments, this creates a region of electric field gradient (typically from the low electric field of the LE to the high electric field of the TE). Analyte ions in the TE overtake TE ions but cannot overtake LE ions and accumulate (“focus” or form a “focused zone”) at the interface between TE and LE. Alternately, target ions in the LE are overtaken by the LE ions; and also accumulate at interface. With judicious choice of LE and TE chemistry, ITP is fairly generally applicable, can be accomplished with samples initially dissolved in either or both the TE and LE electrolytes, and may not require very low electrical conductivity background electrolytes.

As used herein, the term “leading electrolyte” refers to ions having a higher effective electrophoretic mobility as compared to that of the sample ion of interest and/or the trailing electrolyte as used during isotachophoresis. Non-limiting examples of leading electrolytes include chloride, sulfate, and formate.

As used herein, the phrase “next generation sequencing (NGS)” refers to sequencing technologies having high-throughput sequencing as compared to traditional Sanger- and capillary electrophoresis-based approaches, wherein the sequencing process is performed in parallel, for example producing thousands or millions of relatively small sequence reads at a time. Some examples of next generation sequencing techniques include, but are not limited to, sequencing by synthesis, sequencing by ligation, and sequencing by hybridization. These technologies produce shorter reads (anywhere from about 25 to about 500 bp) but many hundreds of thousands or millions of reads in a relatively short time. The term “next-generation sequencing” refers to the so-called parallelized sequencing-by-synthesis or sequencing-by-ligation platforms currently employed by Illumina (e.g., the iSEQ, MiniSEQ, MiSEQ, NextSEQ, NoveSEQ sequencing platforms), and Thermo Fisher Scientific (e.g., the Ion Personal Genome Machine™ (PGM™) System). Next-generation sequencing methods may also include nanopore sequencing methods with electronic-detection (Oxford Nanopore (e.g., MinION, GridION, SmidgION, and PromethION devices) and Roche Diagnostics).

As used herein, the term “nucleic acid” refers to a biochemical macromolecule composed of nucleotide chains that convey genetic information. The most common nucleic acids are deoxyribonucleic acid (DNA) and ribonucleic acid (RNA). The monomers from which nucleic acids are constructed are called nucleotides. Each nucleotide consists of three components: a nitrogenous heterocyclic base, either a purine or a pyrimidine (also known as a nucleobase); and a pentose sugar. Different nucleic acid types differ in the structure of the sugar in their nucleotides; DNA contains 2-deoxyribose while RNA contains ribose.

As used herein, the term “plurality” refers to two or more, for example, 3 or more, 4 or more, 5 or more, etc.

As used herein, a “reaction” between any two different reactive groups (such as any two reactive groups of a reagent and a particle) may mean that a covalent linkage is formed between the two reactive groups (or two reactive functional groups); or may mean that the two reactive groups (or two reactive functional groups) associate with each other, interact with each other, hybridize to each other, hydrogen bond with each other, etc. In some embodiments, the “reaction” includes binding events, e.g., binding events between reactive function groups or binding events between first and second members of a pair of specific binding entities.

As used herein, the term “reagent” refers to solutions or suspensions including one or more agents capable of covalently or non-covalently reacting with, coupling with, interacting with, or hybridizing to another entity. Non-limiting examples of such agents include specific-binding entities, antibodies (primary antibodies, secondary antibodies, or antibody conjugates), nucleic acid probes, oligonucleotide sequences, detection probes, chemical moieties bearing a reactive functional group or a protected functional group, enzymes, solutions or suspensions of dye or stain molecules.

As used herein, the term “sample” refers to biological samples or specimens, environmental samples, and/or samples of synthetic origin (e.g., synthetic polynucleotides, a mixture of molecules following a chemical reaction, etc.). In some embodiments, the sample is a nucleic acid sample which includes one or more target nucleic acids.

As used herein, the term “sequencing” refers to biochemical methods for determining the order of the nucleotide bases, adenine, guanine, cytosine, and thymine, in a DNA oligonucleotide. Sequencing, as the term is used herein, can include without limitation parallel sequencing or any other sequencing method known of those skilled in the art, for example, chain-termination methods, rapid DNA sequencing methods, wandering-spot analysis, Maxam-Gilbert sequencing, dye-terminator sequencing, or using any other modern automated DNA sequencing instruments.

As used herein, the term “specific binding entity” refers to a member of a specific-binding pair. Specific binding pairs are pairs of molecules that are characterized in that they bind each other to the substantial exclusion of binding to other molecules (for example, specific binding pairs can have a binding constant that is at least 10³ M⁻¹ greater, 10⁴ M⁻¹ greater or 10⁵ M⁻¹ greater than a binding constant for either of the two members of the binding pair with other molecules in a biological sample). Particular examples of specific binding moieties include specific binding proteins (for example, antibodies, lectins, avidins such as streptavidins, and protein A). Specific binding moieties can also include the molecules (or portions thereof) that are specifically bound by such specific binding proteins.

As used herein, the term “substantially” means the qualitative condition of exhibiting total or near-total extent or degree of a characteristic or property of interest. In some embodiments, “substantially” means within about 5%. In some embodiments, “substantially” means within about 10%. In some embodiments, “substantially” means within about 15%. In some embodiments, “substantially” means within about 20%.

As used herein, the term “target nucleic acid” refers to a nucleic acid whose presence is to be detected, measured, amplified, and/or subject to further assays and analyses. A target nucleic acid may comprise any single and/or double-stranded nucleic acid. Target nucleic acids can exist as isolated nucleic acid fragments or be a part of a larger nucleic acid fragment. Target nucleic acids can be derived or isolated from essentially any source, such as cultured microorganisms, uncultured microorganisms, complex biological mixtures, biological samples, tissues, sera, ancient or preserved tissues or samples, environmental isolates or the like. Further, target nucleic acids include or are derived from cDNA, RNA, genomic DNA, cloned genomic DNA, genomic DNA libraries, enzymatically fragmented DNA or RNA, chemically fragmented DNA or RNA, physically fragmented DNA or RNA, or the like. In some embodiments, a target nucleic acid may comprise a whole genome. In exemplary embodiments, a target nucleic acid may comprise the entire nucleic acid content of a sample and/or biological sample. In exemplary embodiments, a target nucleic acid may comprise circulating or cell-free DNA's, e.g., circulating tumor DNA (“ctDNA”) present in individuals with cancer or circulating fetal or circulating maternal DNA (“cfDNA”) fragments present in plasma or serum of pregnant women. Target nucleic acids can come in a variety of different forms including, for example, simple or complex mixtures, or in substantially purified forms. For example, a target nucleic acid can be part of a sample that contains other components or can be the sole or major component of the sample. Also a target nucleic acid can have either a known or unknown sequence.

As used herein, the term “trailing electrolyte,” refers to ions having a lower effect electrophoretic mobility as compared to that of the sample ion of interest and/or the leading electrolyte as used during isotachophoresis. Non-limiting examples of trailing electrolytes include IVIES, MOPS, acetate, and glutamate.

Overview

The present disclosure is directed to systems, e.g., automated systems, including an electrophoretic device adapted to facilitate the purification and/or enrichment of a mixture of molecules (e.g., a mixture of nucleic acid molecules). In some embodiments, the systems include an electrophoretic device, one or more downstream processing devices (e.g., sequencing devices, instruments for conducting polymerase chain reactions), and/or one or more feedback control devices, (e.g., chemical analyzers, conductivity detectors), etc. In some embodiments, the systems are suitable for use in sample cleanup and/or target enrichment processes, such as sample cleanup and/or target enrichment processes conducted prior to sequencing a sample using next-generation sequencing. In some embodiments, the systems are suitable for use in sample cleanup and/or target enrichment processes, such as sample cleanup and/or target enrichment processes prior to one or more amplification steps or amplification processes. In some embodiments, the automated systems are also suitable for purifying solutions and/or performing solid-phase synthesis.

Electrophoretic Device

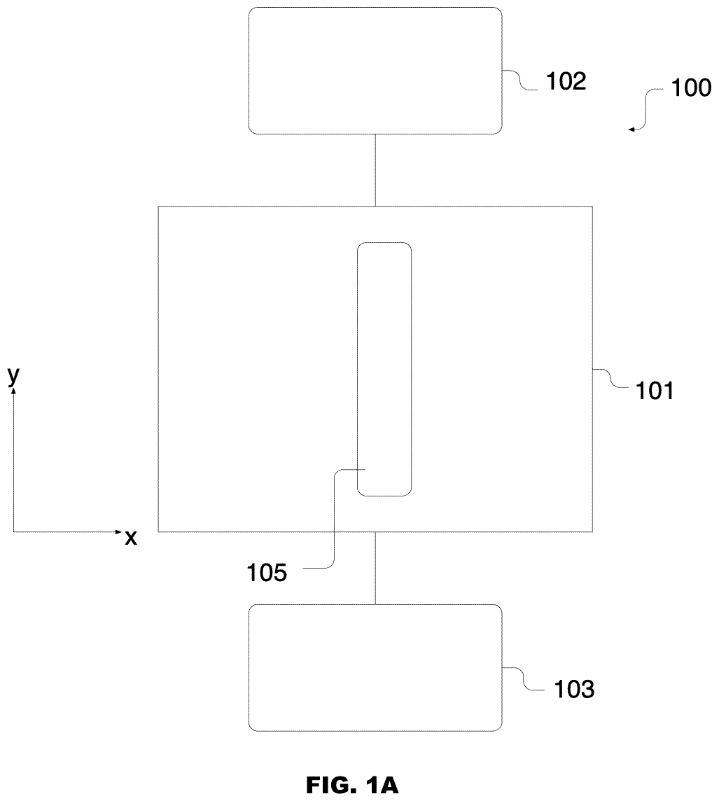

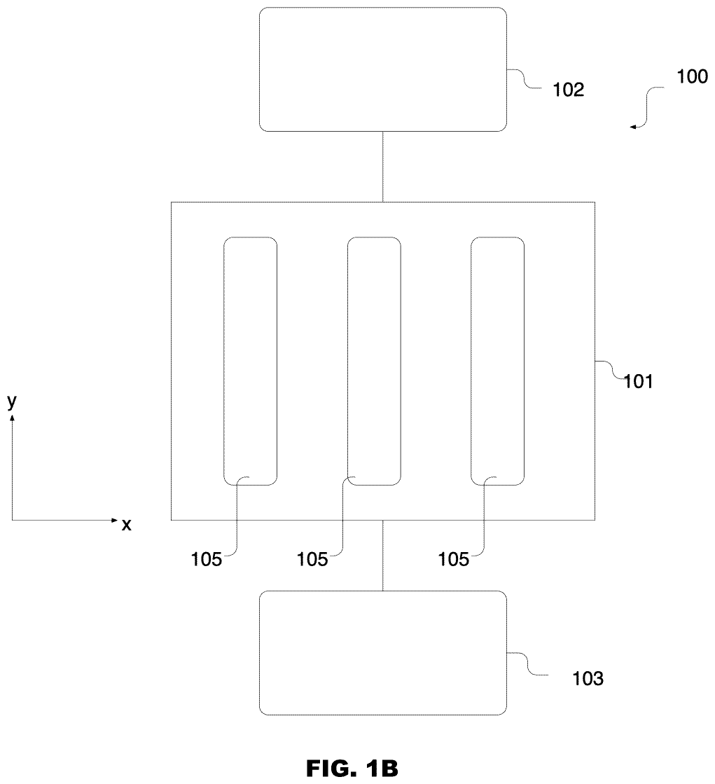

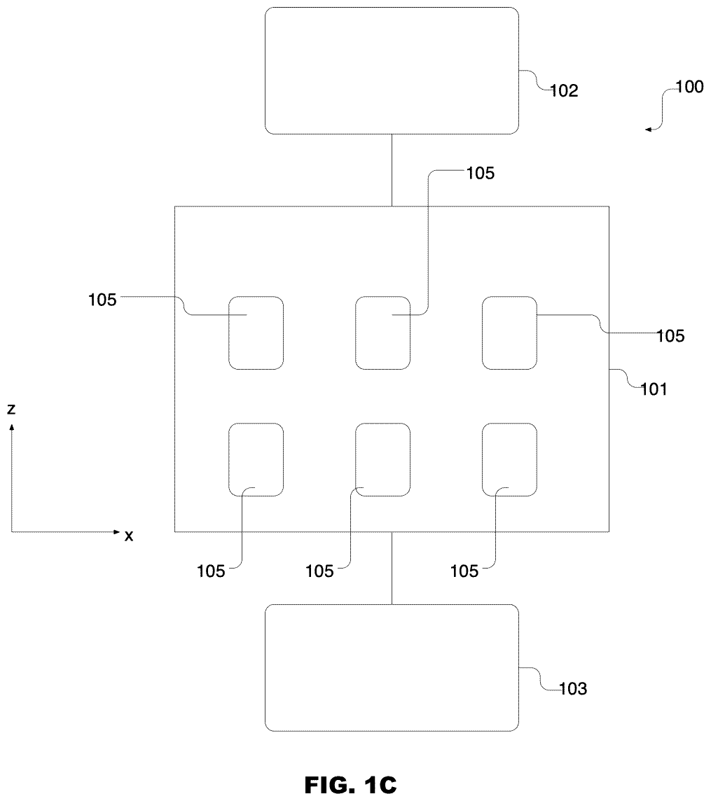

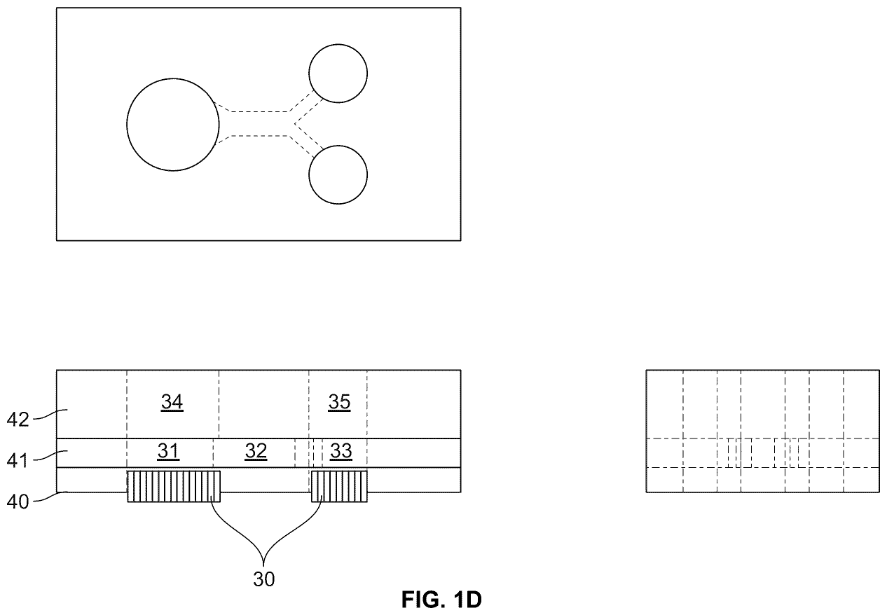

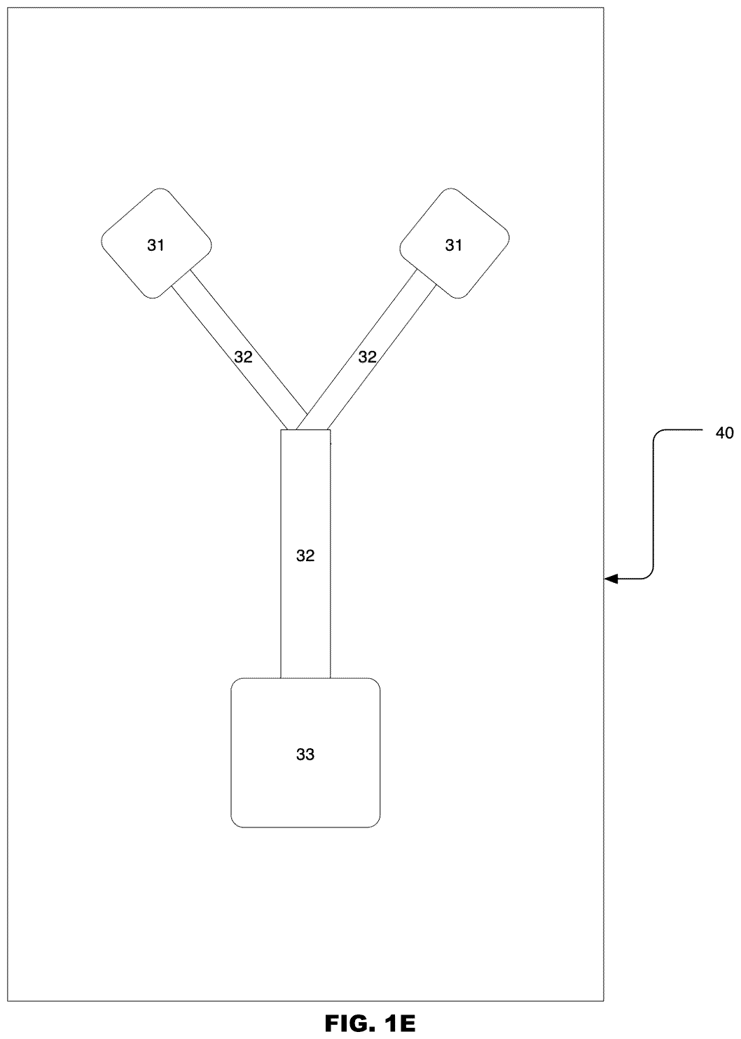

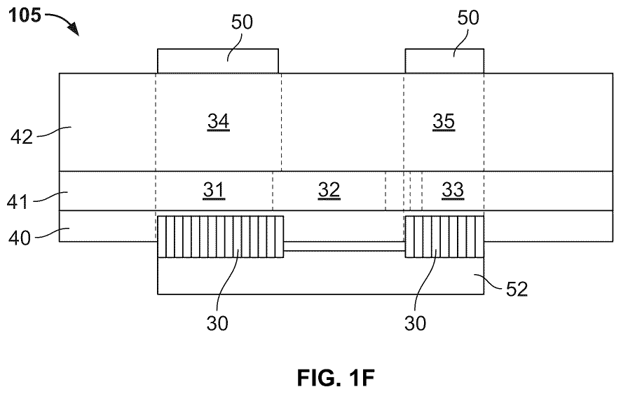

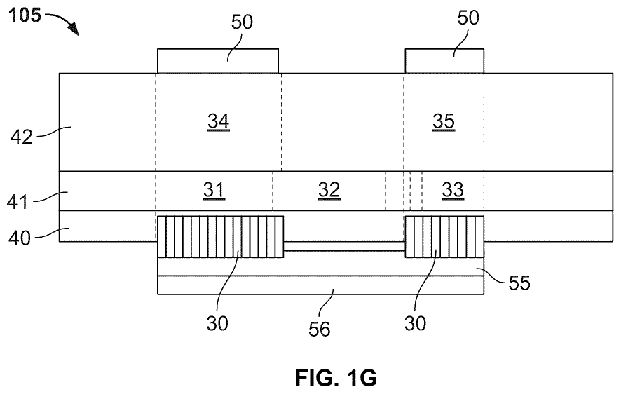

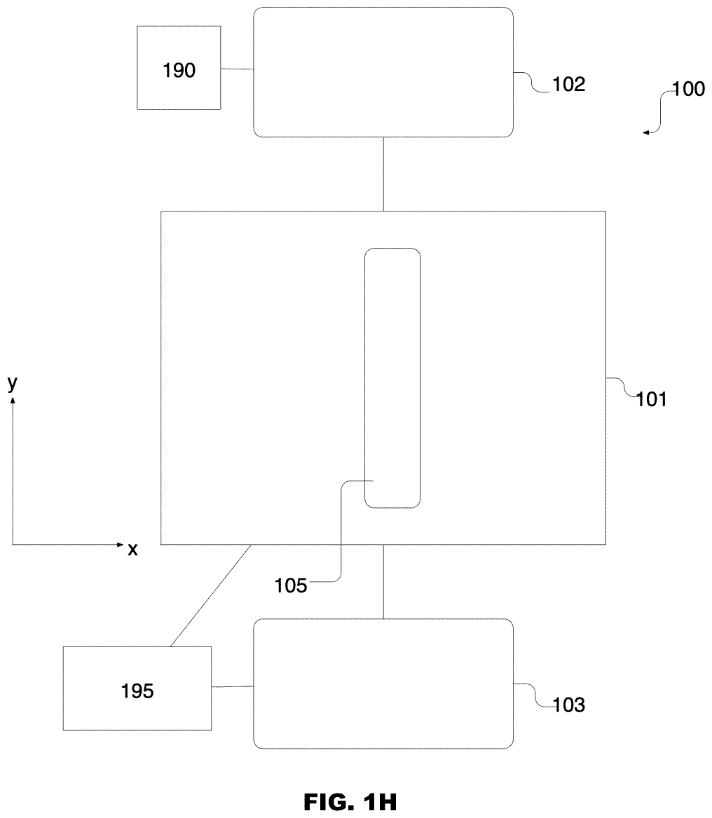

In one aspect of the present disclosure are electrophoretic devices having one or more independently operable separation conduits. With reference to FIGS. 1A-1C, in one aspect of the present disclosure is an electrophoretic device 100 including a fluidics module 102, a control system 103, and an assembly 101, the assembly including one or more independently operable separation conduits 105.

In some embodiments, the one or more independently operable separation conduits 105 are adapted to facilitate the separation or purification of a mixture of molecules. In some embodiments, the one or more independently operable separation conduits 105 are independently operable electrophoretic separation conduits. In some embodiments, the separation and/or purification of molecules within the one or more of the independently operable electrophoretic separation conduits 105 is facilitated via electrophoresis. In some embodiments, the separation and/or purification of molecules within the one or more of the independently operable electrophoretic separation conduits 105 is facilitated via isotachophoresis.

As described in further detail herein, in some embodiments, the electrophoretic devices of the present disclosure include one or more fluid, reagent, or electrolytic material reservoirs. For instance, in some embodiments, each of the one or more independently operable separation conduits of the electrophoretic devices 100 (or any portion thereof) of the present disclosure are fluidically coupled to one or more fluid, reagent, and/or electrolytic material reservoirs. In some embodiments, the electrophoretic devices 100 of the present disclosure further include one or more sensors (e.g., sensors for feedback control) and/or one or more heating and/or cooling modules. Electrophoretic devices 100 and the components constituting any electrophoretic device (e.g., control systems, current/voltage switching systems, pumps, valves, etc.) are described in further detail herein.

Separation Conduits

As noted above, the electrophoretic devices of the present disclosure include one or more independently operable separation conduits. Examples of independently operable separation conduits having different configurations are described herein. While different configurations are described, each of the one or more independently operable separation conduits facilitate the separation and/or purification of molecules within an introduced sample. In some embodiments, each of the one or more independently operable separation conduits may facilitate the separation of a subset or sub-population of molecules from a mixture of molecules including the subset or the sub-population of molecules. For instance, the one or more independently operable separation conduits may facilitate the separation and/or enrichment of target nucleic acids from an introduced mixture of nucleic acids, e.g., an introduced mixture of nucleic acids including both target nucleic acids and non-target nucleic acids.

In some embodiments, a sample is introduced to a sample loading chamber and molecules within the introduced sample may be separated and/or purified by encouraging the molecules to flow through one or more channels and into one or more downstream chambers. In some embodiments, the molecules to be separated and/or purified are encouraged to flow from the sample loading chamber, through the one or more channels, and into the one or more downstream channels through the application of an electric current, voltage, field, or potential. For instance, the molecules to be separated and/or purified are encouraged to flow through the application of an electric field, current, or voltage between: (i) one or more sample loading chambers and one or more waste collection chambers; (ii) one or more sample loading chambers and one or more sample collection chambers; (iii) one or more sample loading chambers and one or more intermediate holding chambers; and (iv) one or more intermediate holding chambers and one or more sample collection chambers. In some embodiments, electrodes are communicatively coupled to each of the channels and/or chambers.

In some embodiments, the molecules to be separated and/or purified are encouraged to flow from the sample loading chamber and through one or more channels pre-loaded with electrically conductive media. In some embodiments, the electrically conductive media is a gel, such as a gel including one or more electroconductive fluids and/or reagents. In some embodiments, the molecules to be separated and/or purified are encouraged to flow through the one or more channels charged with the electroconductive medium such that an introduced sample becomes purified and/or enriched with a subset of the molecules within the introduced sample. In some embodiments, the subset of molecules is suitable for downstream processing, e.g., PCR, sequencing, etc., using one or more downstream processing devices 190 (see, e.g., FIG. 1H). Non-limiting examples of suitable downstream processing devices 190 are described herein.

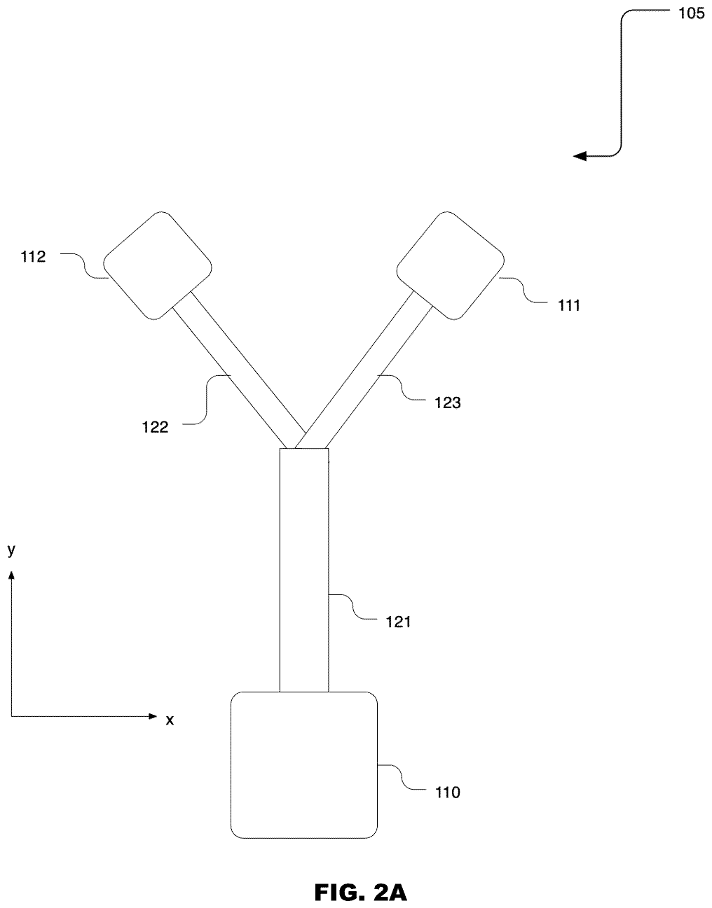

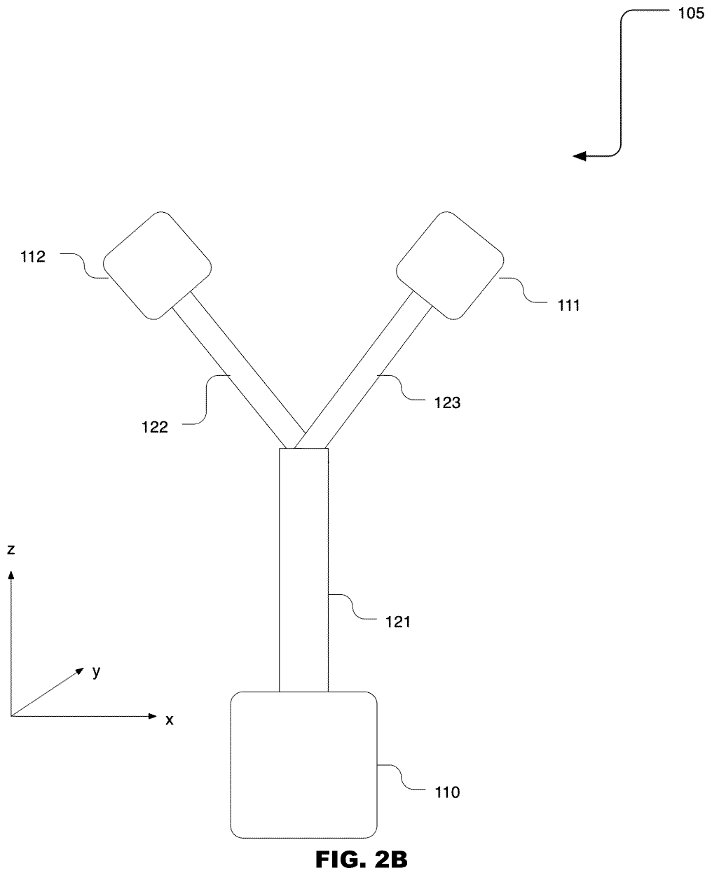

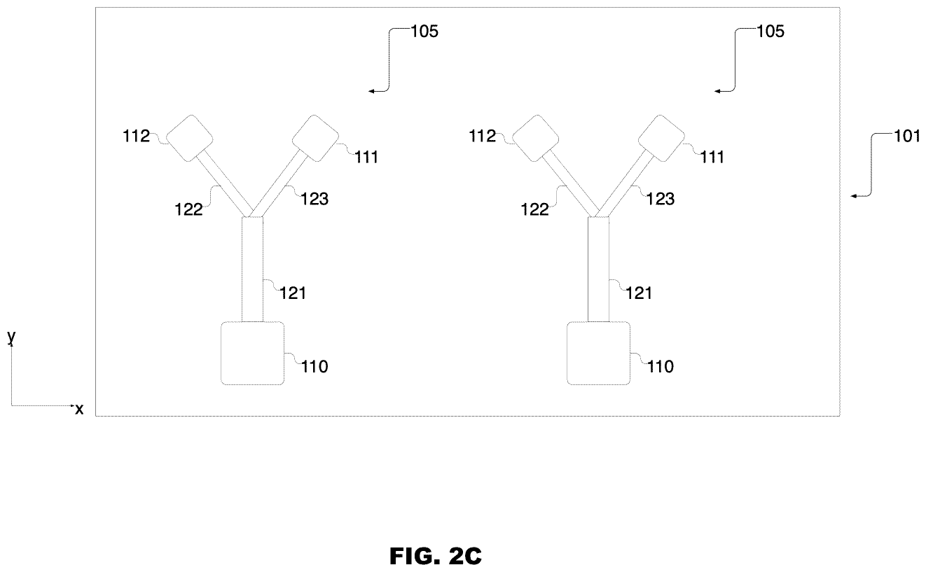

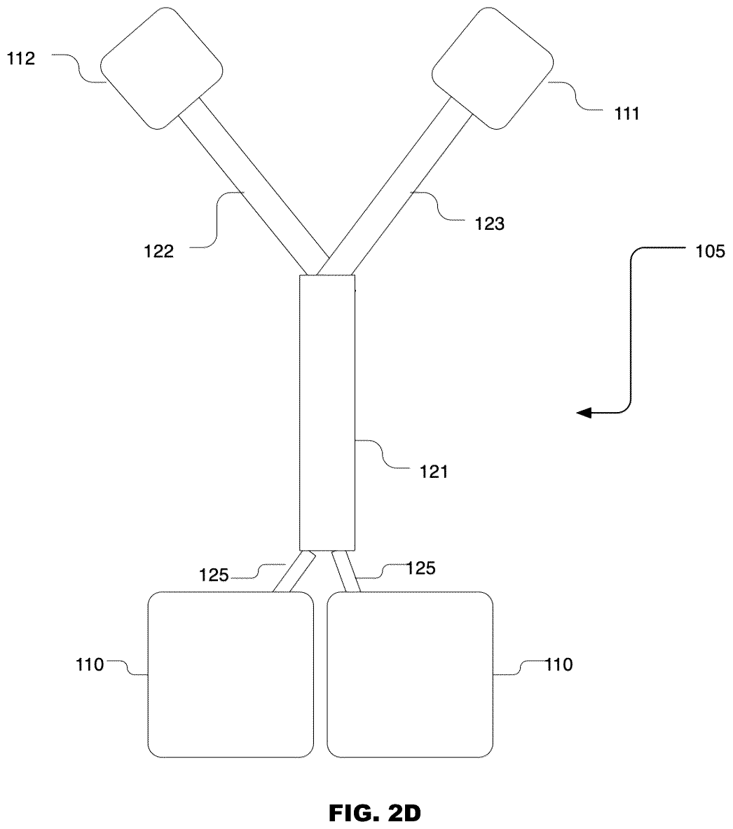

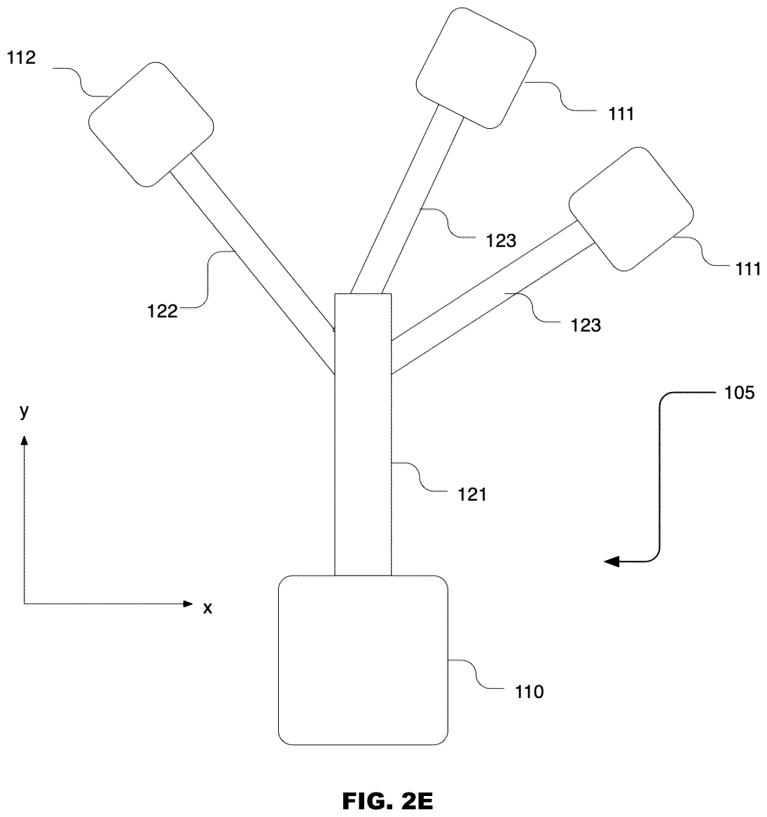

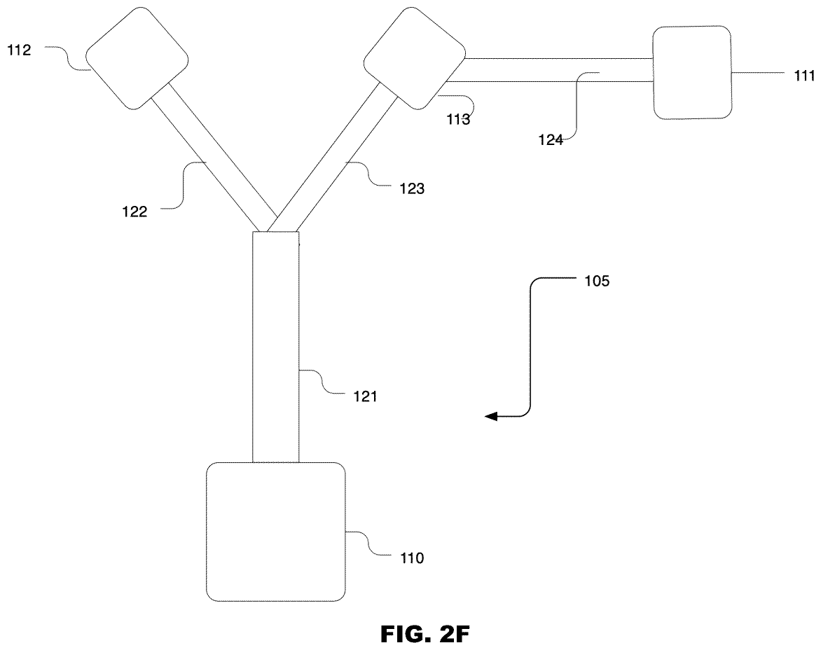

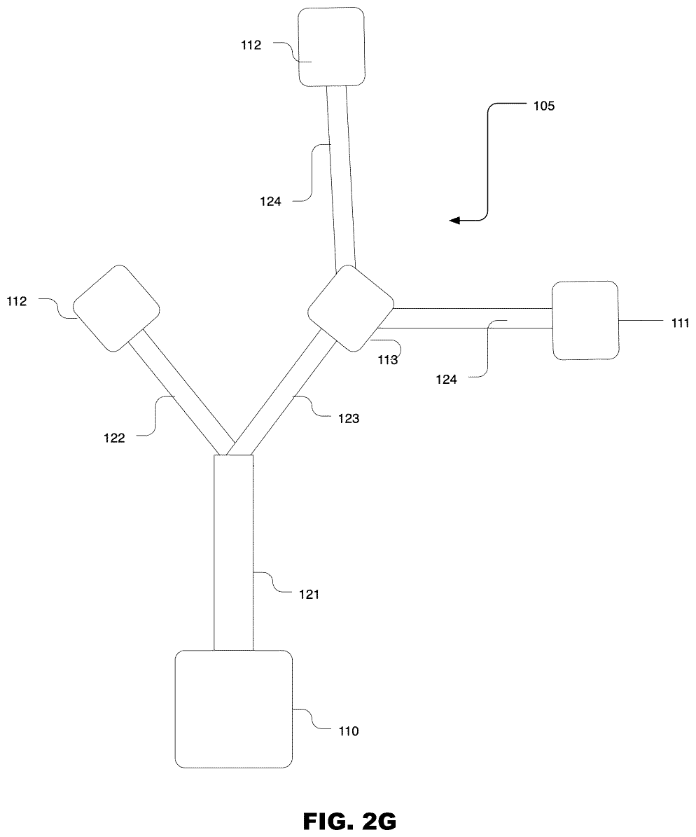

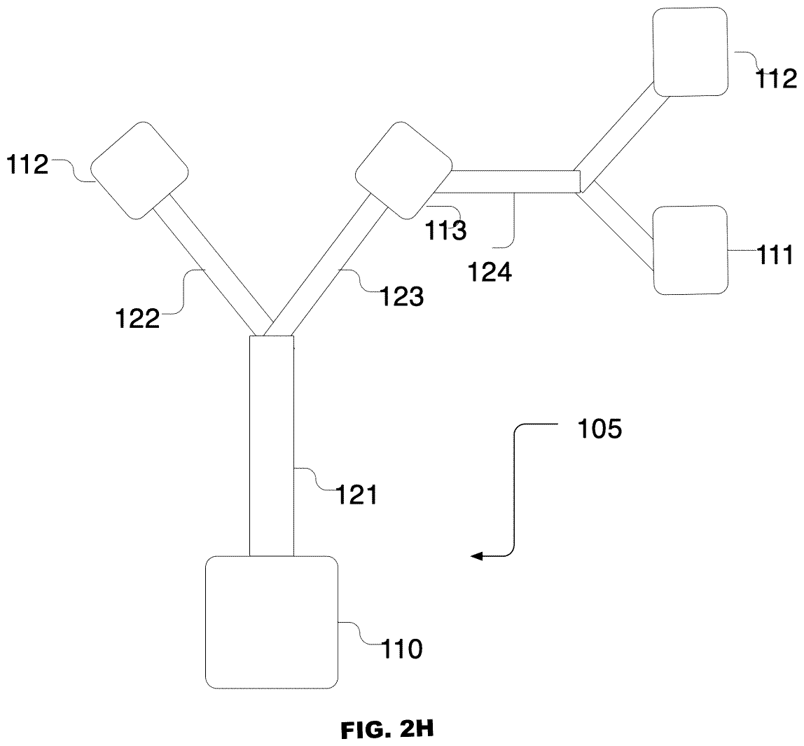

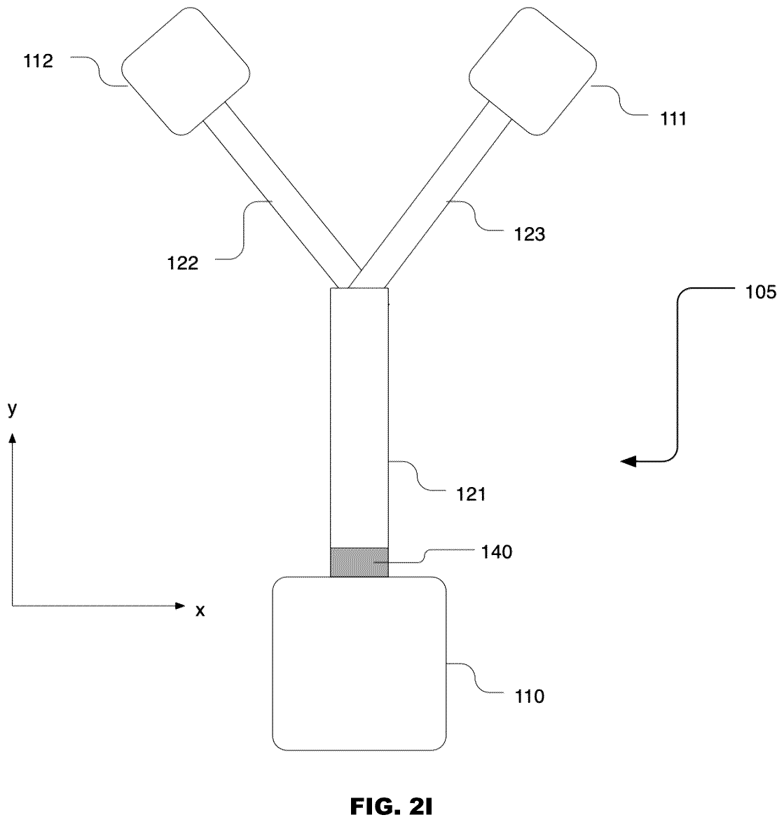

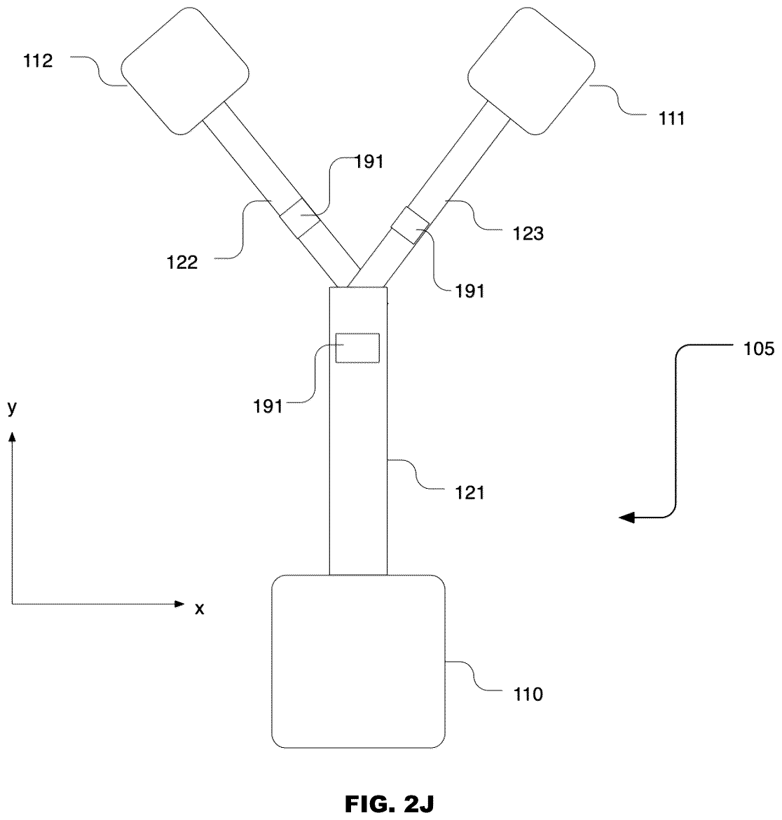

In some embodiments, each of the independently operable separation conduits 105 includes one or more sample loading chambers 110, one or more waste collection chambers 112, and/or one or more sample collection chambers 111 (see, e.g., FIGS. 2A-2E). In other embodiments, each of the independently operable separation conduits 105 includes one or more sample loading chambers 110, one or more waste collection chambers 112, one or more sample collection chambers 111, and one or more intermediate chambers 113 (see, e.g., FIG. 2F). In some embodiments, each of the chambers are in communication with an electrode.

In some embodiments, each independently operable separation conduit 105 includes a single sample loading chamber 110 (see, e.g., FIG. 2A). In other embodiments, each independently operable separation conduit 105 includes two sample loading chambers 110 (see, e.g., FIG. 2D). In yet other embodiments, each independently operable separation conduit 105 includes three sample loading chambers 110. In further embodiments, each independently operable separation conduit 105 includes four or more sample loading chambers 110. In some embodiments, each sample loading chamber is in communication with an electrode.

In some embodiments, each independently operable separation conduit 105 includes a single waste collection chamber 112 (see, e.g., FIGS. 2A-2E). In other embodiments, each independently operable separation conduit 105 includes two waste collection chambers 112. In yet other embodiments, each independently operable separation conduit 105 includes three waste collection chambers 112. In further embodiments, each independently operable separation conduit 105 includes four or more waste collection chambers 112. In some embodiments, each waste collection chamber is in communication with an electrode.

In some embodiments, each independently operable separation conduit 105 includes a single sample collection chamber 111 (see, e.g., FIG. 2A). In other embodiments, each independently operable separation conduit 105 includes two sample collection chambers 111 (see, e.g., FIG. 2E). For instance, there may be two different subsets of target molecules within an introduced sample that are desired to be separated from each other and from non-target molecules and/or impurities. In yet other embodiments, each independently operable separation conduit 105 includes three sample collection chambers 111. In further embodiments, each independently operable separation conduit 105 includes four or more sample collection chambers 111. In some embodiments, each sample collection chamber is in communication with an electrode.

In some embodiments, each independently operable separation conduit 105 includes a single intermediate chamber 113 (see, e.g., FIG. 2F). In other embodiments, each independently operable separation conduit 105 includes two intermediate chambers 113. In yet other embodiments, each independently operable separation conduit 105 includes three intermediate chambers 113. In further embodiments, each independently operable separation conduit 105 includes four or more intermediate chambers 113. In some embodiments, each sample intermediate chamber is in communication with an electrode. In some embodiments, an intermediate chamber may be utilized to conduct a chemical reaction on a first subset of purified molecules, e.g., to further derivative the molecules and/or to add barcodes.

In some embodiments, the one or more sample loading chambers 110, the one or more waste collection chambers 112, the one or more sample collection chambers 111, and/or the one or more intermediate chambers 113 are arranged within the same plane (see, e.g., FIG. 2A, where the chambers are arranged within the same x, y plane). In other embodiments, the one or more sample loading chambers 110, the one or more waste collection chambers 112, the one or more sample collection chambers 111, and/or the one or more intermediate chambers 113 are arranged in different planes, such as stacked vertically (along the z-axis) relative to each other (see, e.g., FIG. 2B).

In some embodiments, each of the one or more sample loading chambers 110, the one or more waste collection chambers 112, the one or more sample collection chambers 111, and/or the one or more intermediate chambers 113 may have any size or shape. In some embodiments, the chambers are substantially circular. In other embodiments, the chambers are ovoid or substantially ovoid. In yet other embodiments, the chambers are rectangular or substantially rectangular.

Each of the one or more sample loading chambers 110, the one or more waste collection chambers 112, the one or more sample collection chambers 111, and/or the one or more intermediate chambers 113 are in communication with one another, e.g., fluidic communication. In some embodiments, each of the one or more sample loading chambers 110, the one or more waste collection chambers 112, the one or more sample collection chambers 111, and/or the one or more intermediate chambers 113 are in communication with each other via one or more interconnected channels, e.g., a plurality of interconnected channels.

In some embodiments, the interconnected channels are branched channels. For instance, a primary channel (such as one in communication with a sample loading chamber) may branch into one or more secondary channels, where each of the one or more secondary channels (e.g., 2 secondary channels, 3 secondary channels, 4 secondary channels, etc.) may independently be in communication with another chamber. In some embodiments, each of the secondary channels may likewise branch into one or more tertiary channels (e.g., 2 tertiary channels, 3 tertiary channels, 4 tertiary channels, etc.), where each of the tertiary channels may independently be in communication with a chamber. In some embodiments, each separation conduit comprises a single branched conduit, e.g., a branched conduit having a “Y” shape.

In some embodiments, the sample loading chamber 110 is in communication with a loading channel 121. In some embodiments, the loading channel 121 is directly coupled to the sample loading chamber 110 (see, e.g., FIGS. 2A, 2B, 2F, and 2I). In other embodiments, the loading channel 121 is indirectly coupled to the sample loading chamber 110. For example, in some embodiments, the loading channel 121 is coupled to one or more inlet channels 125, where each inlet channel 125 is coupled to a sample loading chamber 110 (see, e.g., FIG. 2D).

In some embodiments, the loading channel 121 is in communication with at least two other channels. In some embodiments, the loading channel 121 is in communication with two other channels (see, e.g., FIG. 2A). In other embodiments, the loading channel 121 is in communication with three other channels (see, e.g., FIG. 2E). For example, and as depicted in FIGS. 2A, 2B, 2F, and 2I, a waste collection channel 122 and a sample collection channel 123 are interconnected to and in fluidic communication with the loading channel 121. While each of FIGS. 2A, 2B, 2F, and 2I depict interconnected channels having a substantially “Y” shape, the waste collection channel 122 and the sample collection channel 123 collection channel may be oriented at any angle relative to the loading channel 121 (e.g., an angle ranging from 60° to 120° relative to the sample loading channel 121).

In some embodiments, an intermediate chamber 113 is in communication with an intermediate channel 124 (see, e.g., FIG. 2F). In some embodiments, the intermediate chamber 113 is in communication with one intermediate channel 124 (see, e.g., FIG. 2F). In other embodiments, an intermediate chamber 113 is in communication with at least two intermediate channels 124 (see, e.g., FIG. 2G).

In some embodiments, the intermediate channel 124 is unbranched and coupled to a waste collection chamber 112 or a sample collection chamber 111. For instance, and as illustrated in FIG. 2F, an intermediate chamber 113 is in communication with a sample collection chamber 111 through an unbranched intermediate channel 124. By way of another example, and as illustrated in FIG. 2G, the intermediate chamber 113 is in communication with (i) a sample collection chamber 111 through a first unbranched intermediate channel 124, and (ii) with a waste collection chamber 112 through a second unbranched intermediate channel 124. In yet other embodiments, the intermediate chamber 113 is coupled to a branched intermediate channel 124, whereby the branched intermediate channel 124 is fluidically coupled to two downstream collection chambers, e.g., a waste collection chamber 112 and a sample collection chamber 111 (see, e.g., FIG. 2H).

In some embodiments, the sample loading chamber is configured to include a plurality of beads (e.g., pre-loaded with a plurality of beads); or configured such that a plurality of beads may be introduced into and later removed from the sample loading chamber. In some embodiments, the beads are magnetic beads. In other embodiments, the beads are non-magnetic beads. Examples of suitable non-magnetic beads include silica beads, alginate hydrogel beads, agarose hydrogel beads, poly(N-isopropylacrylamide) (NIPAM) gel beads, cellulose beads, polyethylene (PE) beads, polypropylene (PP) beads, polymethyl methacrylate (PMMA) beads, nylon (PA) beads, polyurethane beads, acrylates copolymer beads, polyquaterniums beads, polysorbate beads, and polyethylene glycol (PEG) beads (any of which may be functionalized or further derivatized before or after introduction to a chamber). In some embodiments, the beads have an average diameter ranging from between about 0.1 μm to about 5 mm. In some embodiments, the beads have an average diameter ranging from between about 0.1 μm to about 4 mm. In some embodiments, the beads have an average diameter ranging from between about 0.1 μm to about 3 mm. In some embodiments, the beads have an average diameter ranging from between about 0.1 μm to about 2 mm. In some embodiments, the beads have an average diameter ranging from between about 0.1 mm to about 1 mm. In yet other embodiments, the beads have an average diameter ranging from between about 0.5 mm to about 1 mm.

In some embodiments, the sample loading chamber 110 is adapted such that any beads introduced to the sample loading chamber 110 may move within the sample loading chamber but not leave the sample loading chamber. In some embodiments, a filter or mesh screen 140 is provided between the sample loading chamber 110 and the loading channel 121 (see, e.g., FIG. 2I), or between the sample loading chamber 110 and one or more inlet channels 125 (not depicted). In some embodiments, the filter or mesh screen allows for molecules to flow out of the sample loading chamber 110 and into the inlet channels 125 and/or the loading channel 121 but prevent beads or other functionalized materials from flowing out of the sample loading chamber 110. Said another way, the filter or mesh screen allows for beads and/or functionalized materials to be substantially retained with the sample loading chamber 110 even after molecules are encouraged to flow from the sample loading chamber 110 and into the loading channel 124. In some embodiments, an opening in the filter or mesh screen ranges from about 0.1 μm to about 1 μm. In other embodiments, an opening in the filter or mesh screen ranges from about 1 μm to about 10 μm. In yet other embodiments, an opening in the filter or mesh screen ranges from about 10 μm to about 100 μm.

In other embodiments, the sample loading chamber 110 is adapted such that any beads introduced to the sample loading chamber 110 may move within the sample loading chamber but not leave the sample loading chamber. In some embodiments, the beads are magnetic beads and one or more magnets may be positioned beneath the sample loading chamber 110 such that the beads are retained within the sample loading chamber 110. In some embodiments, the one or more magnets positioned beneath the sample loading chamber 110 are independently movable, such that the beads are movable within the sample loading chamber 110 while being retained within the sample loading chamber.

In some embodiments, the beads within a sample loading chamber 110 may be retained within the sample loading chamber 110 by including a gel (e.g., agarose (for instance agarose having a concentration of less than about 2%), PAGE) or other separation matrices (such as cellulose derivatives, dextrans, poly(ethylene oxides), and polyvinyl alcohols) within the sample loading channel 121.

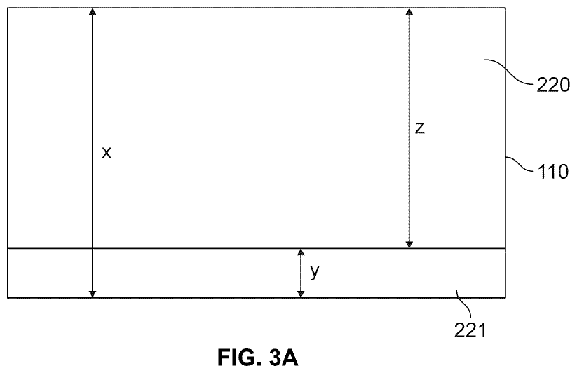

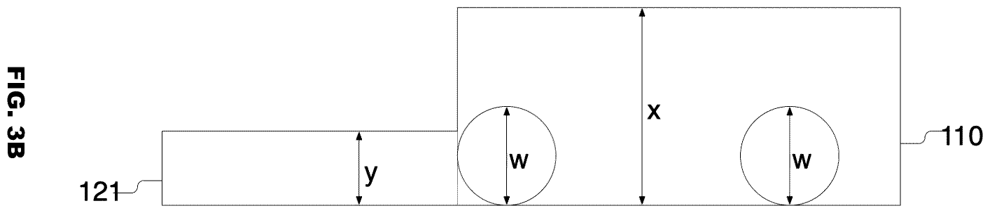

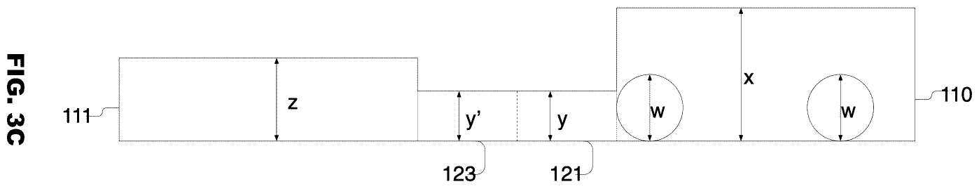

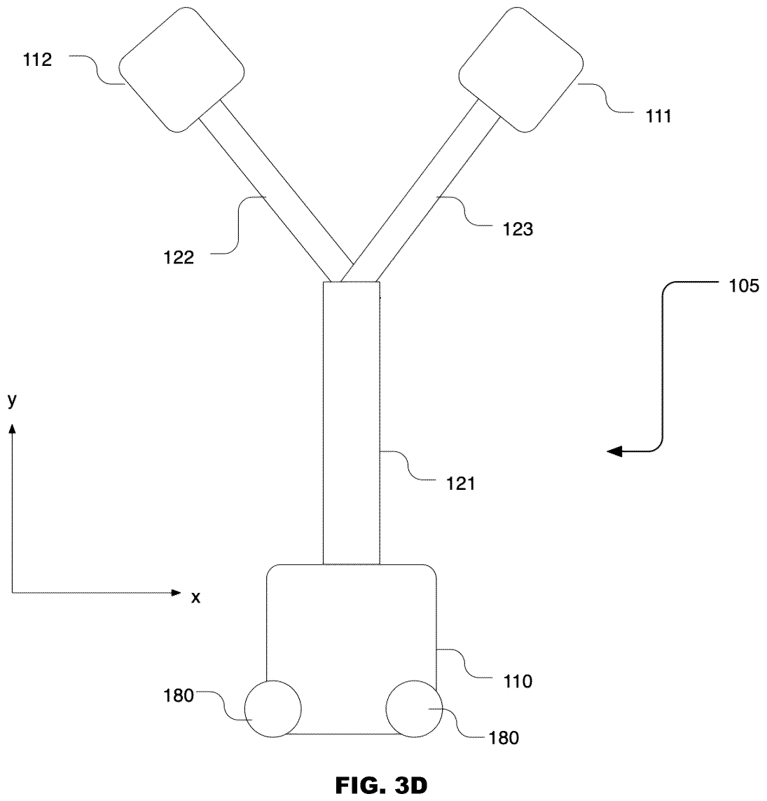

In some embodiments, a wall of the sample loading chamber 110 includes one or more apertures and through which the molecules to be separated and/or purified may be encouraged to flow, but where the beads introduced into the sample loading chamber 110 are retained. FIGS. 3A and 3B illustrate cross-sectional views of a sample loading channel 110. FIG. 3B depicts a plurality of beads 230 each having an average diameter “w,” which is less than a height “x” of wall 220, but greater than a height “y” of aperture 221. In this manner, the molecules to be separated and/or purified may be encouraged to flow through the sample loading chamber 110 and into the loading channel 121, but where a plurality of beads 230 introduced to the sample loading chamber 110 are retained within the sample loading chamber 110 during the or more molecules to be separated and/or purified.

With reference to FIG. 3C, in some embodiments, a height of a wall of a sample loading chamber 110 may be greater than a height of a loading channel 124. For example, and as illustrated in FIG. 3C, a height “x” of the sample loading chamber 110 may be greater than a height “y” of either the sample loading channel 121 or a height “y” of the sample collection channel 123. Likewise, a height “z” of the sample collection chamber 111 may be greater than a height “y” of either the sample loading channel 121 or a height “y” of the sample collection channel 123. In other embodiments, at height of a chamber is less than a height of a channel, provided that when a height of a sample loading chamber 110 is less than a height of a sample loading channel 121, a filter or mesh 140 is provided between the sample loading chamber 110 and the sample loading channel 121 to prevent beads introduced to the sample loading chamber 110 from moving out of the sample loading chamber 110.

In some embodiments, a height “x” of the sample loading chamber 110 ranges from between about 0.1 μm to about 10 cm. In other embodiments, a height “x” of the sample loading chamber 110 ranges from between about 0.1 mm to about 1 cm. In yet other embodiments, a height “x” of the sample loading chamber 110 ranges from between about 0.1 mm to about 1 mm. In some embodiments, a height “y” of a loading channel 121 ranges from between about 10 μm to about 100 μm. In other embodiments, a height “y” of a loading channel 121 ranges from between about 0.1 mm to about 10 mm.

In some embodiments, an area of a bottom surface of the sample loading chamber 110 ranges from between about 1 mm² to about 100 cm². In some embodiments, an area of a bottom surface of the sample loading chamber 110 ranges from between about 1 cm² to about 100 cm². In some embodiments, an area of a bottom surface of the sample loading chamber 110 ranges from between about 1 cm² to about 50 cm². In other embodiments, an area of a bottom surface of the sample loading chamber 110 ranges from between about 1 mm² to about 500 mm². In other embodiments, an area of a bottom surface of the sample loading chamber 110 ranges from between about 1 mm² to about 100 mm². In some embodiments, the sample loading chamber 110 is configured to hold at least 10⁴ beads, at least 10⁵ beads, at least 10⁶ beads, at least 10⁶ beads, at least 10⁷ beads, at least 10⁸ beads, at least 10⁹ beads, at least 10¹⁰ beads, at least 10¹¹ beads, at least 10¹² beads, etc.

In some embodiments, a volume of any chamber, i.e., any of the sample loading, waste collection, intermediate, and/or sample collection chamber, ranges from between about 0.1 μL to about 10 mL. In other embodiments, a volume of a chamber ranges from between about 0.1 μL to about 7.5 mL. In other embodiments, a volume of a chamber ranges from between about 0.1 μL to about 5 mL. In yet other embodiments, a volume of a chamber ranges from between about 0.1 μL to about 2.5 mL. In yet other embodiments, a volume of a chamber ranges from between about 0.1 μL to about 1 mL. In yet other embodiments, a volume of a chamber ranges from between about 0.1 μL to about 0.5 mL.

In some embodiments, each of the chambers employed within a separation conduit 105 have approximately similar volumes. In other embodiments, the sample loading chamber 110 has a comparatively larger volume than either the waste collection chamber 112, the sample collection chamber 111, and/or the intermediate chamber 113. In some embodiments, the sample loading chamber has a volume which is at least 25% larger than a waste collection chamber, a sample collection chamber, and/or an intermediate chamber. In other embodiments, the sample loading chamber has a volume which is at least 50% larger than a waste collection chamber, a sample collection chamber, and/or an intermediate chamber. In yet other embodiments, the sample loading chamber has a volume which is at least 65% larger than a waste collection chamber, a sample collection chamber, and/or an intermediate chamber. In some embodiments, the sample loading chamber has a volume which is at least 80% larger than a waste collection chamber, a sample collection chamber, and/or an intermediate chamber. In some embodiments, the sample loading chamber has a volume which is at least 90% larger than a waste collection chamber, a sample collection chamber, and/or an intermediate chamber. In some embodiments, the sample loading chamber has a volume which is at least 100% larger than a waste collection chamber, a sample collection chamber, and/or an intermediate chamber. In some embodiments, the sample loading chamber has a volume which is at least 150% larger than a waste collection chamber, a sample collection chamber, and/or an intermediate chamber. In some embodiments, the sample loading chamber has a volume which is at least 200% larger than a waste collection chamber, a sample collection chamber, and/or an intermediate chamber. In some embodiments, the sample loading chamber has a volume which is at least 250% larger than a waste collection chamber, a sample collection chamber, and/or an intermediate chamber. In some embodiments, the sample loading chamber has a volume which is at least 300% larger than a waste collection chamber, a sample collection chamber, and/or an intermediate chamber. In some embodiments, the sample loading chamber has a volume which is at least 400% larger than a waste collection chamber, a sample collection chamber, and/or an intermediate chamber. In some embodiments, the sample loading chamber has a volume which is at least 500% larger than a waste collection chamber, a sample collection chamber, and/or an intermediate chamber. In some embodiments, the sample loading chamber has a volume which is at least 600% larger than a waste collection chamber, a sample collection chamber, and/or an intermediate chamber. In some embodiments, the sample loading chamber has a volume which is at least 700% larger than a waste collection chamber, a sample collection chamber, and/or an intermediate chamber. In some embodiments, the sample loading chamber has a volume which is at least 800% larger than a waste collection chamber, a sample collection chamber, and/or an intermediate chamber. In some embodiments, the sample loading chamber has a volume which is at least 900% larger than a waste collection chamber, a sample collection chamber, and/or an intermediate chamber. In some embodiments, the sample loading chamber has a volume which is at least 1000% larger than a waste collection chamber, a sample collection chamber, and/or an intermediate chamber.

In some embodiments, the sample loading chamber 110 is configured to permit the introduction and/or removal of a plurality of beads. In some embodiments, the sample loading chamber 110 may be in fluidic communication with one or more ducts which facilitate the introduction and/or removal of the plurality of beads from the chamber. In some embodiments, the sample loading chamber 110 is in fluidic communication with two ducts. In other embodiments, the sample loading chamber 110 is in fluidic communication with three or more ducts. In yet other embodiments, the sample loading chamber 110 is in fluidic communication with four or more ducts.

FIG. 3D depicts a sample loading chamber 110 in fluidic communication with two ducts 180, where the two ducts 180 are arranged substantially parallel from one another. In some embodiments, one of the two ducts 180 is configured to allow for the introduction of beads while the other of the two ducts 180 is configured to allow for the removal of beads. In some embodiments, each of the ducts 180 may be in fluidic communication with a bead transfer conduit, a bead source (such as a bead storage vessel or a bead collection vessel), one or more valves, and/or one or more pumps.

In some embodiments, the ducts 180 may be external to the sample loading chamber 110. In some embodiments, a wall 200 of the sample loading chamber 110 may include a ductal opening which permits passage of the beads from the one or more ducts 180 into the sample loading chamber 110. In some embodiments, the ductal opening may have any size and/or shape provided that it allows at least one bead to pass into or out of the chamber.

Assemblies

The electrophoretic devices 100 of the present disclosure include an assembly 101 including one or more of the separation conduits 105 described herein. In some embodiments, the assembly 101 includes between 1 and 200 independently operable separation conduits 105. In other embodiments, the assembly 101 includes between 1 and 150 independently operable separation conduits 105. In yet other embodiments, the assembly 101 includes between 1 and 100 independently operable separation conduits 105. In further embodiments, the assembly 101 includes between 1 and 50 independently operable separation conduits 105. In even further embodiments, the assembly 101 includes between 1 and 25 independently operable separation conduits 105. In yet further embodiments, the assembly 101 includes between 1 and 10 independently operable separation conduits 105.

In some embodiments, the assembly 101 includes a single separation conduit 105 (see, e.g., FIGS. 1A and 1H). In other embodiments, the assembly 101 includes two or more independently operable separation conduits 105 (see, e.g., FIG. 2C). In yet other embodiments, the assembly 101 includes three or more independently operable separation conduits 105 (see, e.g., FIG. 1B). In further embodiments, the assembly 101 includes five or more independently operable separation conduits 105. In yet further embodiments, the assembly 101 includes ten or more independently operable separation conduits 105.

In those embodiments where two or more independently operable separation conduits 105 are included within any assembly 101, the independently operable separation units 105 may be arranged within the same plane (see, e.g., FIGS. 1A, 1B, and 2C). By way of example, FIG. 1B illustrates three independently operable separation conduits 105 arranged parallel to one another and within the same plane. In other embodiments, two or more separation conduits 105 may be arranged in different planes. For example, in some embodiments, two or more independently operable separation conduits 105 may be at least partially stacked over each other within any assembly 101 (see, for example, FIG. 1C).

The assembly 101 including the one or more separation conduits 105 may be fabricated of any material suitable for forming a channel and/or conduit. Non-limiting examples of materials include polymers (e.g., polyethylene, polystyrene, polymethylmethacrylate, polycarbonate, poly(dimethylsiloxane), PTFE, PET, and a cyclo-olefin copolymer), glass, quartz, and silicon. The material forming the assembly 101 and any associated components (e.g., a cover) may be hard or flexible. Those of ordinary skill in the art can readily select suitable material(s) based upon e.g., its rigidity, its inertness to (e.g., freedom from degradation by) a fluid to be passed through it, its robustness at a temperature at which a particular device is to be used, its transparency/opacity to light (e.g., in the ultraviolet and visible regions), and/or the method used to fabricate features in the material. For instance, for injection molded or other extruded articles, the material used may include a thermoplastic (e.g., polypropylene, polycarbonate, acrylonitrile-butadiene-styrene, nylon 6), an elastomer (e.g., polyisoprene, isobutene-isoprene, nitrile, neoprene, ethylene-propylene, hypalon, silicone), a thermoset (e.g., epoxy, unsaturated polyesters, phenolics), or combinations thereof.

In some embodiments, the assembly 101 comprises a stack of layers, where the stack of layers may be used to form the chambers and/or channels of each of the independently operable separation conduits 105. In some embodiments, multiple plates or sheets (e.g., polymeric sheets) may be cut (e.g., using a laser cutter) and can be assembled and/or laminated using a double-sided adhesive to create an assembly 101 including the one or more separation conduits 105. In some embodiments, the plates or sheets may be plastics, such as polycarbonate, acryl, polypropylene, etc.

In some embodiments, such as depicted in FIG. 1D, the assembly may comprise a cover layer 42 deposited on a basin layer 41; where the basin layer 41 is deposited on a bottom layer 40. In some embodiments, the bottom layer 40 includes a pre-patterned surface including patterned chambers 31 and 33, and one or more patterned channels 32 (see, e.g., FIG. 1E which depicts a top-down view of a bottom layer 40 having patterned chambers 31 and 33 and one or more patterned channels 32). In some embodiments, the patterned chambers 31 and 33 are in fluidic communication with the one or more patterned channels 32. In some embodiments, the basin layer 41 comprises pre-formed wells 34 and 35, where the pre-formed wells 34 and 35 each approximate the size and shape of the patterned chambers 31 and 33, and where the pre-formed wells 34 and 35 are in fluidic communication with the patterned chambers 31 and 33, respectively. Together, the pre-formed wells 34 and 35 and the patterned chambers 31 and 33 respectively form, by way of example, the sample loading chamber 110 and the waste collection chamber 112.

The electrophoretic devices can also be fabricated by injection molding of thermoplastics such as polycarbonate, polypropylene, and polystyrene. The electrophoretic devices can also be fabricated by 3D printing such as fused deposition modeling (FDM), stereolithography (SLA), selective laser sintering (SLS).

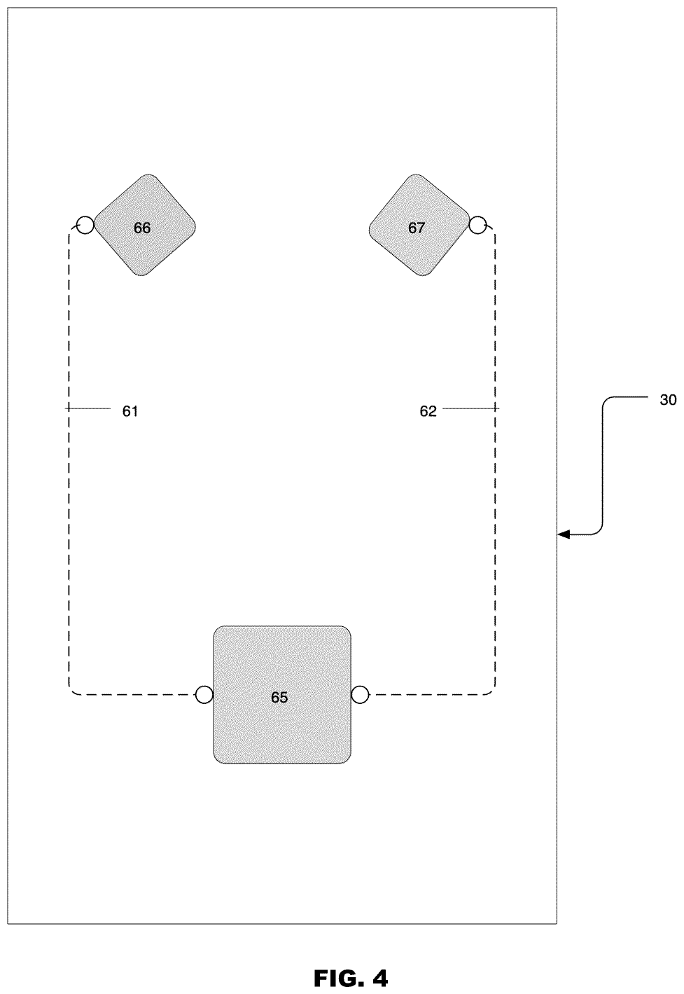

In some embodiments, the chambers within each of the independently operable separation conduits are each independently in electrical communication with one or more electrodes. In some embodiments, electrodes may be shared between any two chambers. In that regard, an assembly 101 may include between 1 and about 500 electrodes, depending on the number of independently operable separation conduits 105 present within the assembly 101. With reference to FIG. 4, electrodes 65, 66, and 67 of electronic layer 30 may each independently be in electrical communication with a different chamber of a separation conduit 105. For example, electrode 65 may be in electrical communication with a sample loading chamber 110; electrode 66 may be in electrical communication with a waste collection chamber 112; and electrode 67 may be in electrical communication with a sample collection chamber 111.

In some embodiments, pairs of any two electrodes may be in communication with each other such that an electric field may be established between any two electrodes and/or any two chambers. For instance, a wire or trace 61 may be used to establish an electric field between electrodes 65 and 66. Likewise, a wire or trace 62 may be used to establish an electric field between electrodes 65 and 67 (see, e.g., FIG. 4).

The conductive layer may include any number of electrodes and/or wires or traces, such that an electric field may be established between any two chambers such that molecules may be encouraged to flow from one chamber to another. In some embodiments, a sample collection chamber and a sample loading chamber may be communicatively coupled together such that an electric field may be established between the sample collection chamber and the sample loading chamber. Likewise, in some embodiments, a waste collection chamber and a sample loading chamber may be communicatively coupled together such that an electric field may be established between the waste collection chamber and the sample loading chamber.

While FIG. 1D illustrates an electronic layer 30 which may include one or more wires or traces and/or electrodes, the present disclosure also contemplates that an electric field may be generated between any two chambers provided that the two chambers are each in communication with an electrode and where each electrode is communicatively coupled together, such as wired together.

Reservoirs and Vessels

The electrophoretic devices 100 of the present disclosure may include one or more reservoirs and/or vessels. In some embodiments, the one or more reservoirs and/or vessels are reagent reservoirs, bead storage vessels, bead collection vessels, fluid reservoirs, waste collection reservoirs, sample collection reservoirs, etc. In some embodiments, the reagent reservoirs, bead storage vessels, bead collection vessels, fluid reservoirs, waste collection reservoirs, sample collection reservoirs are fluidically coupled to one or more separation conduits 105, either directly or indirectly.

The skilled artisan will appreciate that, in some embodiments, each independently operable separation conduit 105 may be coupled to its own set of reagent reservoirs, bead storage vessels, bead collection vessels, fluid reservoirs, waste collection reservoirs, sample collection reservoirs, etc. In other embodiments, two or more of the independently operable separation conduits 105 may be coupled to shared reagent reservoirs, bead storage vessels, bead collection vessels, fluid reservoirs, waste collection reservoirs, sample collection reservoirs, etc. As described herein, fluids, reagents, and/or electrolytic materials from shared reservoirs may be supplied to each independently operable separation conduit 105 by controlling one or more valves disposed within the reservoirs themselves or within conduits coupling the reservoirs to the independently operable separation conduits 105. Likewise, fluids, reagents, and/or electrolytic materials from shared reservoirs may be supplied to the independently operable separation conduits through the action of one or more pumps in fluidic communication with each independently operable separation conduit 105.

In some embodiments, the electrophoretic device 100 includes a separate fluid and/or reagent reservoir for each different fluid and/or reagent to be used in conjunction with the one or more independently operable separation conduits 105. As noted herein, the reservoirs may be shared among two or more independently operable separation conduits 105. For instance, electrolyte reservoirs may be shared between any two or more of the independently operable separation conduits 105.

In some embodiments, the volume of a fluid and/or reagent reservoir ranges from between about 10 μL to about 100 mL. In some embodiments, the volume of a fluid and/or reagent reservoir ranges from between about 10 μL to about 50 mL. In some embodiments, the volume of a fluid and/or reagent reservoir ranges from between about 10 μL to about 10 mL. In some embodiments, the volume of a fluid and/or reagent reservoir ranges from between about 1 mL to about 5 mL.

In some embodiments, each independently operable separation conduit 105 may be fluidically coupled to one or more bead storage or collection vessels. In some embodiments, beads may be introduced to sample loading chambers of a separation conduit 105 through a conduit coupling a bead storage vessel to a first duct in communication with the sample loading chamber of the separation conduit 105. Likewise, in some embodiments, beads may be transferred from a sample loading chamber of a separation conduit 105 through a conduit coupling a bead collection vessel to a second duct in communication with the sample loading chamber of the separation conduit 105.

In some embodiments, the volume of a bead storage or collection vessel ranges from between about 0.1 μL to about 50 mL. In other embodiments, the volume of bead storage or collection vessel ranges from between about 0.1 mL to about 10 mL. In other embodiments, the volume of bead storage or collection vessel ranges from between about 0.1 mL to about 5 mL. In some embodiments, beads may be introduced into the separation conduit 105 of the electrophoretic device 100 through a bead packing inlet connected to a bead storage vessel using a pipette or syringe.

Fluidics Module

The electrophoretic devices 100 of the present disclosure may also include one or more fluidics modules. In some embodiments, each of the one or more fluidics modules may include one or more conduits, one or more pumps, one or more valves, etc.

Conduits

The electrophoretic device 100 may include any number of conduits to facilitate the transfer of fluids, reagents, electrolytic materials, and/or beads to chambers and/or channels of the separation conduit 105 of the electrophoretic device 100. In some embodiments, each fluid and/or reagent for introduction to the separation conduit 105 (or a component thereof) may be stored in a separate fluid reservoir and/or reagent reservoir and wherein each fluid reservoir and/or reagent reservoir is independently coupled to a fluid transfer conduit or a reagent transfer conduit in fluidic communication with a chamber or channel of the separation conduit 105. In this manner, a reagent from a single reagent reservoir may be transferred via a reagent transfer conduit to a chamber or channel of each independently operable separation conduit 105. Likewise, a fluid from a single fluid reservoir may be transferred via a fluid transfer conduit to a chamber or channel of each independently operable separation conduit 105. In some embodiments, each of the fluid and/or reagent reservoirs and/or the fluid and/or reagent transfer conduits may include a valve, e.g. a 2-way valve, such that fluids and/or reagents may be flowed into the chambers and channels of each independently operable separation conduit 105, as described herein.

For example, in some embodiments, a first conduit may be used to transfer a sample including one or more populations of molecules from a sample loading chamber to a sample collection chamber such that the one or more populations of molecules may be separated and/or purified. In some embodiments, a second conduit may be used to transfer a population of separated molecules from a sample collection conduit to a downstream processing device 190, e.g., a sequencing device.

Valves

The electrophoretic device 100 of the present disclosure may include one or more valves and/or microvalves. In some embodiments, the valves may be disposed within any conduit of the electrophoretic device 100, with any portion of a conduit of the electrophoretic device 100, or at a junction of any two conduits of the electrophoretic device 100. In some embodiments, each of the valves of the electrophoretic device 100 includes one or more ports, e.g., 1-port, 2-ports, or 3-ports.

Any type of valve may be utilized provided that the valve allows the flow of fluid, reagents, and/or beads throughout the electrophoretic device 100 to be regulated, e.g., starting/stopping fluid flow, controlling the quantities of fluid flow, etc. In some embodiments, the valves are controlled based on signals from a control system 103, e.g., the control system 103 may command a valve to actuate to a first position, to a second position, or a third position such that fluid, reagent, and/or bead transfer may be regulated. Non-limiting examples of suitable microfluidic valves are described in U.S. Pat. No. 10,197,188; in U.S. Patent Publication Nos. 2008/0236668 and 2006/0180779; and in PCT Publication No. WO/2018/104516, the disclosures of which are hereby incorporated by reference herein in their entireties.

In some embodiments, one or more valves may be disposed in each fluid transfer conduit or a reagent transfer conduit such that the flow of fluids and/or reagents from the reservoirs may be independently controlled. Alternatively, in other embodiments, each of the fluid and/or reagent reservoirs include a valve such that the flow of fluids and/or reagents from the reservoirs may be independently controlled.

Pumps

In some embodiments, the electrophoretic device 100 is in fluidic communication with one or more pumps. In some embodiments, the electrophoretic device is in fluidic communication with two pumps. In other embodiments, the electrophoretic device is in fluidic communication with three pumps. In yet other embodiments, the electrophoretic device is in fluidic communication with four or more pumps.

In some embodiments, the one or more pumps facilitate the movement of fluid, reagents, samples, and/or beads to and from the reservoirs, chambers, and/or conduits of the independently operable separation conduits 105 of the electrophoretic device 100. Any pump may be utilized within the electrophoretic device 100 of the present disclosure provided that the pump allows for control of the volume of materials loaded into or discharged from the components of electrophoretic device 100. In some embodiments, the one or more pumps are pressure pumps. In other embodiments, the one or more pumps are piezo-electric pumps. In some embodiments, the one or more pumps are peristaltic pumps. In some embodiments, the one or more pumps are syringe pumps. In some embodiments, the one or more pumps are volumetric pumps.

In some embodiments, the one or more pumps of the present disclosure have a volume ranging from between about 1 mL to about 100 mL. In other embodiments, the one or more pumps of the present disclosure have a volume ranging from between about 10 mL to about 100 mL. In some embodiments, the one or more pumps of the present disclosure may deliver a flow rate of between about 1 μL/minute to about 1000 mL/minute. In other embodiments, the one or more pumps of the present disclosure may deliver a flow rate of between about 10 μL/minute to about 500 mL/minute. In yet other embodiments, the one or more pumps of the present disclosure may deliver a flow rate of between about 10 μL/minute to about 100 mL/minute.

In some embodiments, the one or more pumps are one or more micropumps. In some embodiments, the one or more micropumps are mechanical pumps (e.g., diaphragm micropumps and peristaltic micropumps). In some embodiments, the one or more micropumps are non-mechanical pumps (e.g., valveless micropumps, capillary pumps, and chemically powered pumps). Micropumps and other devices for pumping small fluid quantities are described, for example, in U.S. Pat. Nos. 5,094,594, 5,730,187 and 6,033,628, each of which disclose devices which can pump fluid volumes in the nanoliter or picoliter range (the disclosures of which are hereby incorporated by reference herein in their entireties).

Other pumps suitable for use with the electrophoretic devices 100 of the present disclosure are described in U.S. Pat. No. 10,208,739; and in U.S. Publication Nos. 2015/0050172 and 2017/0167481, the disclosures of which are each hereby incorporated by reference herein in their entireties.

Control System and Other Modules

The presently disclosed electrophoretic devices 100 include a control system 103. The control system 103, in some embodiments, includes one or more memories and one or more programmable processors. In some embodiments, in order to store information the control system 103 may include, without limitation, one or more storage elements, such as volatile memory, non-volatile memory, read-only memory (ROM), random access memory (RAM), or the like. In some embodiments, the control system 103 is a stand-alone computer, which is external to the system. The storage and/or memory device can be one or more physical apparatuses used to store data or programs on a temporary or permanent basis. In some instances, the device is volatile memory and requires power to maintain stored information. In other instances, the device is non-volatile memory and retains stored information when the digital processing device is not powered. In still other instances, the non-volatile memory comprises flash memory. The non-volatile memory can comprise dynamic random-access memory (DRAM). The non-volatile memory can comprise ferroelectric random-access memory (FRAM). The non-volatile memory can comprise phase-change random access memory (PRAM). The device can be a storage device including, by way of non-limiting examples, CD-ROMs, DVDs, flash memory devices, magnetic disk drives, magnetic tapes drives, optical disk drives, and cloud computing based storage.

In some embodiments, the control system 103 is a networked computer which enables control of the system remotely. The term “programmed processor” encompasses all kinds of apparatus, devices, and machines for processing data, including by way of example a programmable microprocessor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

In some embodiments, the control system 103 is in electrical communication with the one or more electrodes, traces, and/or wires such that an electric field may be established between any two chambers of a separation conduit, e.g., between a sample collection chamber and a sample loading chamber, or between a waste collection chamber and a sample loading chamber. In some embodiments, the control system 103 is operable to switch “on” or “off” an electric field between any two chambers of the separation conduit 105, e.g., between a sample collection chamber and a sample loading chamber, or between a waste collection chamber and a sample loading chamber. In other embodiments, the control system 103 is operable to regulate the electric field (e.g., the strength of the field) supplied between any two chambers of the separation conduit 105, e.g., between a sample collection chamber and a sample loading chamber, or between a waste collection chamber and a sample loading chamber.

In some embodiments, the control system 103 is used to send instructions to the various pumps and/or valves (such as those included within a fluidics module 102) so as to regulate a flow of a material into or out of any one of the chambers of a separation conduit or any of the reservoirs of the electrophoretic device (e.g., into or out of a sample loading chamber). In some embodiments, the control system 103 is configured to send instructions to actuate one or more valves to open or close, including one or more valves disposed in a reservoir and/or in a conduit. In some embodiments, the control system is configured to send instructions to regulate the operation of one or more pumps, such as to cause the one or more pumps to introduce or withdraw fluids, reagents, and/or beads from one or more of the reservoirs and/or chambers of each independently operable separation conduit 105.

In some embodiments, the electrophoretic devices 100 (or any component thereof) may be coupled to one or more feedback control devices 195 (see, e.g., FIG. 1H). In some embodiments, feedback control involves the detection of one or more events or processes occurring in the presently disclosed electrophoretic devices 100 or within any one of the independently operable separation conduits 105. In some embodiments, feedback control may involve, for example, the determination of electrical conductivity, electrical resistivity, electrical current, electrical voltage, temperature, fluorescence signal intensity, chromatic intensity or Raman signal intensity. In that regard, in some embodiments non-limiting examples of feedback control devices 195 which may be used to monitor separation and/or facilitate feedback control include one or more of a conductivity detector 191 (see, e.g., FIG. 2J), a multimeter, a fluorescent camera, a microscope, a machine vision camera, a thermal imaging camera, and/or a Raman spectrometer.

In some embodiments, the one or more feedback control devices 195 include one or more sensors 50, such as one or more pressure sensors, temperature sensors and/or flow rate sensors which may be monitored by the control system 103, such as in real-time (see, e.g., FIGS. 1F and 1G).

In some embodiments, the one or more feedback control devices 195 are chemical analyzers. In some embodiments, the chemical analyzers may be used to detect cellular components, reagents, byproducts, etc. within a collected fluid stream (e.g., a waste stream). In some embodiments, the chemical analyzers are selected from Qubit for nucleic acid quantification Bioanalyzer for size distribution of NAs, Lightcycler 480 qPCR instrument for nucleic acid quantification, mass spectrometers such as MALDI-TOF MS, LC/MS/MS, CE-MS etc. for molecular identification and/or quantification. In some embodiments, the one or more feedback control devices 195 are optical microscopes (bright field, fluorescent). In some embodiments, a fluorescence microscopy device may include one or more laser sources and CCD and/or CMOS-based imaging sensors and/or cameras. In some embodiments, the one or more feedback control devices 195 are spectrometers (IR, NMR, Raman).

In some embodiments, the one or more feedback control devices 195 may be coupled to the control system 103 to permit feedback control of the electrophoretic device 100. In some embodiments, the control system 103 is configured to receive data from one or more feedback control devices 195, process the received data, and regulate a temperature, a reaction condition, etc. based on the received and processed data. By way of example, the control system 103, in some embodiments, is configured to execute a series of instructions to control or operate one or more system components to perform one or more operations, e.g., preprogrammed operations or routines, or to receive feedback from one or more one or more feedback control devices 195 communicatively coupled to the system and command the one or more system components to operate (or cease to operate) depending on the feedback received.

In some embodiments, the electrophoretic device 100 includes one or more thermal regulation modules 52, e.g., one or heating modules 55 and/or cooling modules 56 (see, e.g., FIGS. 1F and 1G). In some embodiments, the assembly, separation conduits, reagent reservoirs, fluid reservoirs, and/or any conduits are each independently in thermal communication with a separate thermal regulation module 52. In this manner, each separation conduit 105 (or portions thereof) may be independently heated and/or cooled through the operation of one or more thermal regulation modules 52 in communication therewith. In other embodiments, the assembly, separation conduits, reagent reservoirs, fluid reservoirs, and/or any conduits may be in thermal communication with one or more shared thermal regulation modules.

Suitable thermal regulation modules 52 include heating blocks, Peltier devices, and/or thermoelectric modules. Suitable Peltier devices include any of those described within U.S. Pat. Nos. 4,685,081, 5,028,988, 5,040,381, and 5,079,618, the disclosures of which are hereby incorporated by reference herein in their entireties.

In some embodiments, the control system 103 may be communicatively coupled to the one or thermal regulation modules 52 and configured to command the thermal regulation modules 52 to activate to heat and/or cool the assembly 100, the independently operable separation conduits 105, the reagent reservoirs, the fluid reservoirs, and/or the conduits to a pre-determined temperature for a pre-determined amount of time. For example, a control system 103 may direct heating from at least one thermal regulation module 52 such that a predetermined temperature is reached within the assembly 100 and/or maintained within the assembly 100 for a predetermined amount of time. The predetermined temperature may be input to the control system 103 by a user or may be provided in the form of pre-programmed instructions or routines.

Downstream Processing Devices

In some embodiments, the electrophoretic devices 100 of the present disclosure may be coupled to one or more downstream processing devices 190, including, but not limited to, sequencing devices and/or devices for conducting polymerase chain reaction. In some embodiments, the one or more downstream processing devices 190 are one or more sequencing devices. In some embodiments, the sequencing device is a “next generation sequencing” device. The term “next generation sequencing” refers to sequencing technologies having high-throughput sequencing as compared to traditional Sanger- and capillary electrophoresis-based approaches, wherein the sequencing process is performed in parallel, for example producing thousands or millions of relatively small sequence reads at a time. Some examples of next generation sequencing techniques include, but are not limited to, sequencing by synthesis, sequencing by ligation, and sequencing by hybridization. These technologies produce shorter reads (anywhere from about 25 bp-about 500 bp) but many hundreds of thousands or millions of reads in a relatively short time.

Examples of such sequencing devices available from Illumina (San Diego, CA) include, but are not limited to iSEQ, MiniSEQ, MiSEQ, NextSEQ, NoveSEQ. It is believed that the Illumina next-generation sequencing technology uses clonal amplification and sequencing by synthesis (SBS) chemistry to enable rapid sequencing. The process simultaneously identifies DNA bases while incorporating them into a nucleic acid chain. Each base emits a unique fluorescent signal as it is added to the growing strand, which is used to determine the order of the DNA sequence.

A non-limiting example of a sequencing device available from ThermoFisher Scientific (Waltham, MA) includes the Ion Personal Genome Machine™ (PGM™) System. It is believed that Ion Torrent sequencing measures the direct release of H+ (protons) from the incorporation of individual bases by DNA polymerase. A non-limiting example of a sequencing device available from Pacific Biosciences (Menlo Park, CA) includes the PacBio Sequel System. A non-limiting example of a sequencing device available from Roche (Pleasanton, CA) is the Roche 454.

Next-generation sequencing methods may also include nanopore sequencing methods. In general, three nanopore sequencing approaches have been pursued: strand sequencing in which the bases of DNA are identified as they pass sequentially through a nanopore, exonuclease-based nanopore sequencing in which nucleotides are enzymatically cleaved one-by-one from a DNA molecule and monitored as they are captured by and pass through the nanopore, and a nanopore sequencing by synthesis (SBS) approach in which identifiable polymer tags are attached to nucleotides and registered in nanopores during enzyme-catalyzed DNA synthesis. Common to all these methods is the need for precise control of the reaction rates so that each base is determined in order.

Strand sequencing requires a method for slowing down the passage of the DNA through the nanopore and decoding a plurality of bases within the channel; ratcheting approaches, taking advantage of molecular motors, have been developed for this purpose. Exonuclease-based sequencing requires the release of each nucleotide close enough to the pore to guarantee its capture and its transit through the pore at a rate slow enough to obtain a valid ionic current signal. In addition, both of these methods rely on distinctions among the four natural bases, two relatively similar purines and two similar pyrimidines. The nanopore SBS approach utilizes synthetic polymer tags attached to the nucleotides that are designed specifically to produce unique and readily distinguishable ionic current blockade signatures for sequence determination.

In some embodiments, sequencing of nucleic acids via nanopore sequencing comprises preparing nanopore sequencing complexes and determining polynucleotide sequences. Methods of preparing nanopores and nanopore sequencing are described in U.S. Patent Application Publication No. 2017/0268052, and PCT Publication Nos. WO2014/074727, WO2006/028508, WO2012/083249, and WO/2014/074727, the disclosures of which are hereby incorporated by reference herein in their entireties. In some embodiments, tagged nucleotides may be used in the determination of the polynucleotide sequences (see, e.g., PCT Publication No. WO/2020/131759, WO/2013/191793, and WO/2015/148402, the disclosures of which are hereby incorporated by reference herein in their entireties).

Analysis of the data generated by sequencing is generally performed using software and/or statistical algorithms that perform various data conversions, e.g., conversion of signal emissions into base calls, conversion of base calls into consensus sequences for a nucleic acid template, etc. Such software, statistical algorithms, and the use of such are described in detail, in U.S. Patent Application Publication Nos. 2009/0024331 2017/0044606 and in PCT Publication No. WO/2018/034745, the disclosures of which are hereby incorporated by reference herein in their entireties.

In some embodiments, the one or more downstream processing devices 190 are one or more devices for conducting polymerase chain reaction (PCR). In general, PCR is a method for increasing the concentration of a segment of a target sequence in a mixture of genomic DNA without cloning or purification. Examples of PCR techniques that can be used include, but are not limited to, quantitative PCR, quantitative fluorescent PCR (QF-PCR), multiplex fluorescent PCR (MF-PCR), real time PCR (RT-PCR), single cell PCR, restriction fragment length polymorphism PCR (PCR-RFLP), PCR-RFLP/RT-PCR-RFLP, hot start PCR, nested PCR, in situ polony PCR, in situ rolling circle amplification (RCA), bridge PCR, picotiter PCR, digital PCR, droplet digital PCR, and emulsion PCR. Polymerase chain reaction (“PCR”) is described, for example, in U.S. Pat. Nos. 4,683,202; 4,683,195; 4,000,159; 4,965,188; 5,176,995), the disclosures of each are hereby incorporated by reference herein in their entirety.

Commercially available droplet and digital droplet PCR systems are available, e.g., from Bio-Rad and ThermoFisher. Descriptions of dPCR can be found, e.g., in US20140242582; Kuypers et al. (2017) J Clin Microbiol 55:1621; and Whale et al. (2016) Biomol Detect Quantif 10:15. Droplet and digital droplet PCR systems are further described in U.S. Pat. Nos. 9,822,393 and 10,676,778, the disclosures of which are hereby incorporated by reference herein in their entireties. In some embodiments, the droplets for digital droplet PCR may be generated by any of the devices described in PCT Application No. WO/2010/036352, the disclosure of which is hereby incorporated by reference herein in its entirety.

Methods

The present disclosure is also directed to methods of purifying a sample, enriching a sample with desired target molecules, and/or performing solid-phase chemical reactions using the electrophoretic devices of the present disclosure. In some embodiments, the methods are carried out in a closed system which mitigates the risk of cross-contamination and/or or sample loss. In some embodiments, the methods described herein reduce the amount of labor-intensive work required. In some embodiments, the methods described herein facilitate sample purification with reduced incubation times as compared to magnetic separation processes. In some embodiments, the methods described herein require no pipetting steps and thus prevent or mitigate bead loss, sample loss, and/or contamination.

In some embodiments, the electrophoretic devices employed herein include one or more independently operable separation conduits, where each of the separation conduits include a plurality of beads (e.g., pre-loaded with a plurality of beads), such as functionalized beads. In some embodiments, the functionalized beads are magnetic functionalized beads. Examples of magnetic beads include Dynabeads, AMPureXP beads, KAPA pure beads. In other embodiments, the functionalized beads are non-magnetic functionalized beads. Suitable non-magnetic beads are described in U.S. Pat. No. 5,328,603, the disclosure of which is hereby incorporated by reference herein in its entirety.

In some embodiments, a surface of the functionalized beads includes a first moiety (e.g., a first reactive functional group) which is reactive with a second moiety (e.g., a second reactive functional group) of a molecule (or a conjugate including the molecule) within the sample to be purified. In some embodiments, a “reaction” between a first moiety and a second moiety may mean that a covalent linkage is formed between two reactive groups or two reactive functional groups of the two moieties; or may mean that the two reactive groups or two reactive functional groups of the two moieties associate with each other, interact with each other, hybridize to each other, hydrogen bond with each other, etc. In some embodiments, the “reaction” thus includes binding events, such as the binding of a hapten with an anti-hapten antibody, or the binding of biotin with streptavidin.

In some embodiments, the functionalized beads may comprise avidin or streptavidin to bind to biotinylated molecules (e.g., molecules conjugated to biotin) within the sample to be purified. In some embodiments, the functionalized beads comprisees immobilized antibodies, which may be used to bind to molecules including or conjugated to specific antigenic molecules. In yet other embodiments, the functionalized beads comprises enzymes, which may be used to bind to molecules including or conjugated to specific enzyme substrates. In further embodiments, the functionalized beads comprises receptors, which may be used to bind to molecules including or conjugated to specific receptor ligands. In yet further embodiments, the functionalized beads comprises lectins, which may be used to bind to molecules including or conjugated to specific polysaccharides. In even further embodiments, the functionalized beads comprises nucleic acids, which may be used to bind to molecules including or conjugated to complementary base sequences. In some embodiments, DNA/RNA aptamers tethered onto the bead surface may specifically bind to its target analytes such as small molecules, peptides, proteins, cells. In other embodiments, molecules including terminal amine moiety may be bound to an NETS-activated surface or a carboxyl-activated surface.

General Method of Purifying a Sample Introduced to an Electrophoretic Device

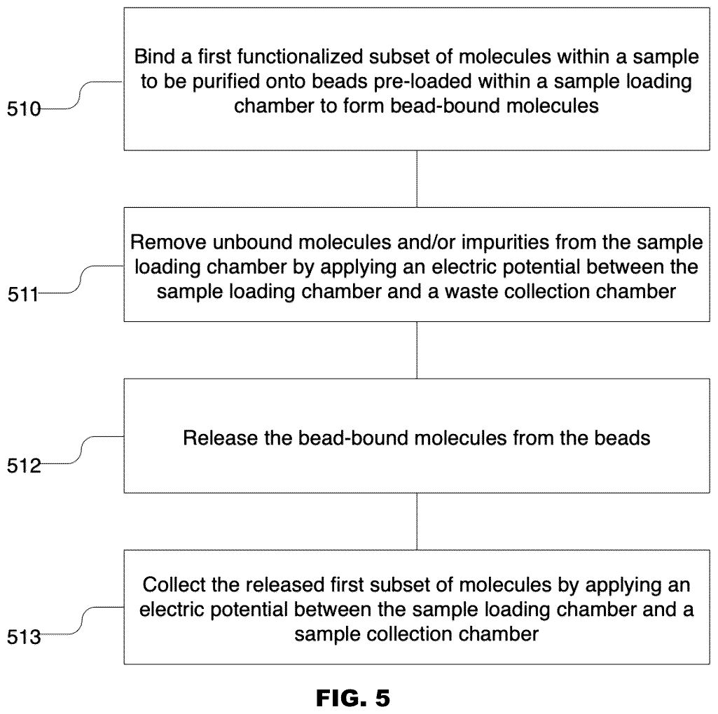

In some embodiments, the present disclosure is directed to methods of purifying a sample using any one of the electrophoretic devices described herein (see, e.g., FIG. 5). For example, the sample may be purified with an electrophoretic device which includes one or more independently operable separation conduits, such as an electrophoretic device including one separation conduit or an electrophoretic device including two or more independently operable separation conduits. In these embodiments, the method of purification does not rely on actively flowing fluids (e.g., such as by pumping) through one or more chambers and/or channels (e.g., where the molecules to be separation and/or purified would be carried or transported by the active fluid flow). Rather, the methods disclosed herein encourage the movement of molecules between chambers of the independently operable separation conduits by establishing an electric field between different chambers of the separation conduit.

In some embodiments, a sample is first introduced to one or more sample loading chambers of a separation conduit of the electrophoretic device. In some embodiments, the sample may be pumped into the one or more sample loading chambers from a reservoir or another upstream vessel or chamber (e.g., an upstream reaction vessel or chamber), such as via one or more conduits in fluidic communication with the one or more sample loading chambers.

In some embodiments, each of the one or more sample loading chambers are pre-loaded with a plurality of beads, such as a plurality of beads each having a functionalized surface. In some embodiments, each of the one or more sample loading chambers are pre-loaded with between about 10 to about 10,000 beads each having a functionalized surface (which may be magnetic or non-magnetic). In other embodiments, each of the one or more sample loading chambers are pre-loaded with between about 10 to about 1000 beads each having a functionalized surface. In yet other embodiments, each of the one or more sample loading chambers are pre-loaded with between about 10 to about 150 beads each having a functionalized surface.

In some embodiments, the sample loading chamber is also pre-loaded with one or more electrolytes. In some embodiments, the sample loading chamber is pre-loaded within between about 1 μL to about 100 μL of the one or more electrolytes. In other embodiments, the sample loading chamber is pre-loaded within between about 0.1 mL to about 10 mL of the one or more electrolytes. In some embodiments, the one or more electrolytes are each trailing electrolytes. In some embodiments, the one or more electrolytes are selected from TAPS-TRIS, Tris/Acetate/EDTA (TAE), and Tris/Borate/EDTA (TBE). In some embodiments, the plurality of beads and the one or more electrolytes are introduced as a mixture to the sample loading chamber.

In some embodiments, other channels within the separation conduit of the electrophoretic device are also pre-loaded with one or more electrolytes. In some embodiments, the other chambers of the separation conduit are pre-loaded within between about 1 μL to about 100 μL of the one or more electrolytes. In other embodiments, the other chambers of the separation conduit are pre-loaded within between about 0.01 mL to about 10 mL mL of the one or more electrolytes. In some embodiments, the one or more electrolytes are each leading electrolytes. In some embodiments, the one or more electrolytes are selected from HCl-Histidine, Tris/Acetate/EDTA (TAE), and Tris/Borate/EDTA (TBE).

In some embodiments, each of a waste collection chamber and a sample collection chamber are pre-loaded within one or more electrolytes, e.g., one or more leading electrolytes. In some embodiments, the one or more electrolytes introduced to the waste collection chamber is the same as those introduced to the sample collection chamber. In other embodiments, the one or more electrolytes introduced to the waste collection chamber are different than those introduced to the sample collection chamber. In yet other embodiments, a concentration of one or more electrolytes introduced to the waste collection chamber is different than a concentration of one or more electrolytes introduced to the sample collection chamber.

In some embodiments, one or more channels in communication with the sample loading chamber and/or the other chambers, e.g., waste collection chamber and/or sample loading chamber, are pre-loaded with a gel. In some embodiments, the gel is agarose and/or PAGE. For instance, a gel may be infused into the one or more channels (or a branched channel having one or more levels of branching) prior to or subsequent to the introduction of the sample into the sample loading chamber. In some embodiments, the gel is pumped into the one or more channels (or branched channel) from a reservoir and through one or more conduits in fluidic communication with the one or more channels (or the various branches of the branched channel). In some embodiments, a gel is formed in situ within the one or more channels (or the branched channel). In some embodiments, the gel serves as a sieving matrix and/or a filter to retain the plurality of beads within the sample loading chamber.

In some embodiments, the sample may include one or more subsets or sub-populations of molecules. In some embodiments, it is desirable to separate a first subset of the one or more subsets of molecules from the remaining subsets of molecules. In some embodiments, the first subset of molecules are functionalized to provide a first subset of functionalized molecules. In some embodiments, the first subset of molecules are functionalized by reacting the first subset of molecules within an appropriate reagent which selectively reacts with only the first subset of molecules in the sample. By way of example, the reagent may be an oligonucleotide (or a pool of different oligonucleotides) that selectively hybridizes with the first subset of molecules, and where the oligonucleotide (or a pool of different oligonucleotides) includes a first reactive moiety capable of reacting with a second reactive moiety, such as a second reactive moiety of a functionalized bead. In this manner, the first subset of molecules becomes functionalized with the first moiety such that it may react with the second moiety of the functionalized bead.

In some embodiments, the first subset of functionalized molecules are generated prior to the input sample's introduction to the separation conduit. For instance, the first subset of molecules may be functionalized in an upstream reaction vessel or chamber and be transferred to the sample loading chamber, with or without any prior purification steps.

In some embodiments, the first moiety may include biotin to bind to a second moiety of a functionalized bead including avidin or streptavidin. In other embodiments, the first moiety may include a thiolated molecule to bind to a second moiety of a functionalized bead which includes gold particles. In yet other embodiments, the first moiety may include an amine-terminated molecule to bind to an NETS-activated substrate.

In some embodiments, the first moiety includes immobilized antibodies, which may be used to bind to a second moiety of a functionalized bead including or conjugated to specific antigenic molecules. In other embodiments, the first moiety includes an antigenic molecule which may be used to bind to a second moiety of a functionalized bead, where the second moiety includes immobilized antibodies.

In some embodiments, the first moiety includes enzymes, which may be used to bind to a second moiety of a functionalized bead including or conjugated to specific enzyme substrates. In other embodiments, the first moiety includes a substrate for an ezynme, which may be used to bind to a second moiety of a functionalized bead, where the second moiety includes an enzyme.

In some embodiments, the first moiety includes receptors, which may be used to bind to a second moiety of a functionalized bead including or conjugated to specific receptor ligands. In other embodiments, the first moiety includes one or more receptor ligands, which may be used to bind to a second moiety of a functionalized bead, where the second moiety includes receptors.

In some embodiments, the first moiety includes lectins, which may be used to bind to a second moiety of a functionalized bead including or conjugated to specifc polysaccharides. In other embodiments, the first moiety includes one or more polysaccarides, which may be used to bind to a second moiety of a functionalized bead, where the second moiety includes or is conjugated to one or more lectins.

In some embodiments, the first subset of functionalized molecules included within the sample introduced to the sample loading chamber binds to the beads pre-loaded within the sample loading chamber to form bead-bound molecules (step 510). In some embodiments, the reaction proceeds without the introduction of any additional fluids and/or reagents. In other embodiments, one or more fluids (e.g., buffers) are introduced with the sample; and/or one or more reagents are introduced with the sample.

In some embodiments, the introduced sample is incubated with the beads for between about 1 minutes to about 1500 minutes. In some embodiments, the introduced sample is incubated with the beads for between about 1 minutes to about 1000 minutes. In some embodiments, the introduced sample is incubated with the beads for between about 1 minutes to about 500 minutes. In some embodiments, the introduced sample is incubated with the beads for between about 1 minutes to about 250 minutes. In some embodiments, the introduced sample is incubated with the beads for between about 1 minutes to about 100 minutes. In other embodiments, the introduced sample is incubated with the beads for between about 5 minutes to about 60 minutes. In some embodiments, incubation takes place at a temperature above room temperature, e.g., at a temperature of at least about 30° C. or greater, at a temperature of at least about 35° C. or greater, at a temperature of at least about 45° C. or greater, at a temperature of at least about 50° C. or greater, etc.

Other subsets of molecules within the introduced sample and which do not include the proper functionalization, i.e., the proper first reactive moiety, do not bind to the beads pre-loaded within the sample loading chamber. Thus, the sample loading chamber will include bead-bound molecules, unbound non-functionalized molecules, and/or impurities.

Following the formation of the bead-bound molecules, the unbound non-functionalized molecules and/or impurities are removed from the sample loading chamber (step 511). In some embodiments, the unbound non-functionalized molecules and/or impurities are transported from the sample loading chamber, through a branched channel, and to another chamber, e.g., a waste collection chamber. This may be accomplished by establishing an electric field between the sample loading chamber and the other chamber of the separation conduit, e.g., a waste collection chamber.

For instance, the unbound molecules and/or impurities are encouraged to move from the sample loading chamber to the waste collection chamber by applying an electric field between the sample loading chamber and the waste collection chamber. In some embodiments, the control system directs a switch to “open” (i.e., opening an electric current pathway) allowing current to flow between a first electrode in communication with the sample loading chamber and a second electrode in communication with the waste collection chamber. For instance, a current established between a sample loading chamber and a waste collection chamber may range from between about 10 mA to about 10 A. In other embodiments, the current established ranges from between 10 mA and 5 A. In other embodiments, the current established ranges from between 10 mA and 1 A. In other embodiments, the current established ranges from between 10 mA and 500 mA. In other embodiments, the current established ranges from between 10 mA and 100 mA. In other embodiments, a voltage established between a sample loading chamber and a waste collection chamber may range from between about 10V to about 10 kV. In other embodiments, a voltage established between a sample loading chamber and a waste collection chamber may range from between about 10V to about 5 kV. In other embodiments, a voltage established between a sample loading chamber and a waste collection chamber may range from between about 10V to about 1 kV. In other embodiments, a voltage established between a sample loading chamber and a waste collection chamber may range from between about 10V to about 500V. In other embodiments, a voltage established between a sample loading chamber and a waste collection chamber may range from between about 10V to about 250V. In other embodiments, a voltage established between a sample loading chamber and a waste collection chamber may range from between about 10V to about 100V.

In some embodiments, the collected unbound non-functionalized molecules and/or impurities may be further transferred from the waste collection chamber to another vessel in communication therewith. For example, the collected unbound non-functionalized molecules and/or impurities transferred to the waste collection chamber may be pumped via a conduit to another vessel.

In some embodiments, the unbound non-functionalized molecules and/or impurities being transferred out of the chamber may be monitored, such as with camera, e.g., a fluorescent camera, to determine if substantially all unbound molecules and/or impurities have been removed. In some embodiments, a fluorescent camera with laser source can be utilized to monitor the sample loading chamber, the transfer channel, and the waste collection chamber, such as for fluorescent signals emitted from unbound molecules and/or impurities, and this signal can be fed back to the control system to command the amount of current flowing between the electrodes in communication with the chambers, or to stop a current flow (i.e., ‘close’ the current flow path). For instance, as molecules are transferred through the channel, a feedback control device (e.g., a fluorescent detector) in communication therewith may be used to detect and/or quantify the unbound molecules and/or impurities within the waste stream.

In other embodiments, the feedback control device is a conductivity detector 191 (such as one in communication with any of the sample loading chamber, the transfer channel, and/or the waste collection chamber, such as depicted in FIG. 2J) may be also used to detect local pH changes caused by a molecular composition locally and again this acquired pH data may be used for feedback control.

In some embodiments, one or more reagents may be optionally introduced to the sample loading chamber to derivatize the bead-bound molecules, e.g., to further react the bead-bound molecule with an introduced reagent, such as with a reagent introduced to the sample loading chamber through a conduit in communication therewith. In some embodiments, impurities and/or excess reagents may be removed by again establishing an electrical field (e.g., an electrical potential) between the sample loading chamber and the waste collection chamber as described above. The process of reacting the one or more bead-bound molecules may be repeated any number of times to synthesize any desired molecules. For instance, the process may be repeated once, two or more times, three or more times, etc. In some embodiments, the reaction allows for the introduction of one or more molecule bar codes, tags, labels (e.g., detectable labels), etc.

Following the removal of the unbound non-functionalized molecules and/or impurities transferred to the waste collection chamber, the bead-bound molecules are released from the beads (step 512). In some embodiments, the bead-bound molecules are chemically released. For example, a fluid and/or a reagent may be introduced to the sample loading chamber to effectuate the release of the bead-bound molecules. In some embodiments, the bead-bound molecules are incubated with a reagent for a pre-determined amount of time. In some embodiments, an incubation period may range from between about 15 seconds to about 90 minutes. In some embodiments, an incubation period may range from between about 1 minute to about 60 minutes. In other embodiments, an incubation period may range from between about 1 minute to about 20 minutes.

In some embodiments, the bead-bound molecules include a chemically labile group. In some embodiments, the bead-bound molecules include a cleavable group. Non-limiting examples of chemically cleavable groups include disulfide-based groups; diazobenzene groups (e.g., 2-(2-alkoxy-4-hydroxy-phenylazo); benzoic acid scaffolds; ester bond-based groups; and acidic sensitive groups (e.g., a dialkoxydiphenylsilane group or acylhydrazone group). Electrophilically cleaved groups (e.g., p-alkoxybenzyl esters and p-alkoxybenzyl amides) are believed to be cleaved by protons and include cleavages sensitive to acids.

In other embodiment, the bead-bound molecules include an enzymatically cleavable group. In some embodiments, the enzymatically cleavable group includes a trypsin cleavable group or a protease cleavable groups. In some embodiments, the group may be enzymatically cleaved by one of an uracil-N-glycosylase, an RNase A, a beta-glucuronidase, a beta-galactosidase, or a TEV-protease. In some embodiments, the bead-bound molecules are incubated with an enzyme for a pre-determined amount of time. In some embodiments, an incubation period may range from between about 15 seconds to about 90 minutes. In some embodiments, an incubation period may range from between about 1 minute to about 60 minutes. In other embodiments, an incubation period may range from between about 1 minute to about 20 minutes.

In other embodiments, the bead-bound molecules are released upon a change in temperature. For instance, the bead-bound molecules may include a thermally labile group. In some embodiments, the control system may command one or more thermal regulation modules to heat the sample loading chamber and the bead-bound molecules therein to a temperature ranging from between about 30° C. to about 95° C. In other embodiments, the control system may command one or more thermal regulation modules module to heat the sample loading chamber and the bead-bound molecules therein to a temperature ranging from between about 40° C. to about 70° C. In other embodiments, the control system may command one or more thermal regulation modules to heat the sample loading chamber and the bead-bound molecules therein to a temperature ranging from between about 45° C. to about 70° C. In other embodiments, the control system may command one or more thermal regulation modules to heat the sample loading chamber and the bead-bound molecules therein to a temperature ranging from between about 47° C. to about 65° C. In yet other embodiments, the control system may command one or more thermal regulation modules to heat the sample loading chamber and the bead-bound molecules therein to a temperature ranging from between about 30° C. to about 95° C. In some embodiments, the control system commands one or more thermal regulation modules in communication with the sample loading chamber to heat the sample loading chamber and/or the bead-bound molecules to a temperature in which two hybridized strands of a nucleic acid molecule become denatured, e.g., 90° C. to about 95° C.

In yet other embodiments, the bead-bound molecules include a photocleavable group. In some embodiments, the photocleavable group is a group which may be cleaved upon exposure to an electromagnetic radiation source having a wavelength of between about 200 nm to about 400 nm (UV) or between about 400 nm to about 800 nm (visible). Examples of suitable photocleavable groups include, but are not limited to, arylcarbonylmethyl groups (e.g., 4-acetyl-2-nitrobenzyl, dimethylphenacyl (DMP)); 2-(alkoxymethyl)-5-methyl-α-chloroacetophenones, 2,5-dimethylbenzoyl oxiranes, benzoin groups (e.g., 3′,5′-dimethoxybenzoin (DMB)), o-nitrobenzyl groups (e.g., 1-(2-nitrophenyl)ethyl (NPE), 1-(methoxymethyl)-2-nitrobenzene, 4,5-dim ethoxy-2-nitrob enzyl (DMNB), α-carboxynitrobenzyl (α-CNB)); o-nitro-2-phenethyloxycarbonyl groups (e.g., 1-(2-nitrophenyl)ethyloxycarbonyl and 2-nitro-2-phenethyl derivatives); o-nitroanilides (e.g., acylated 5-bromo-7-nitroindolines); coumarin-4-yl-methyl groups (e.g., 7-methoxycoumarin derivatives); 9-substituted xanthenes, and arylmethyl groups (e.g., o-hydroxyarylmethyl groups). In even further embodiments, the bead-bound molecules include a photocleavable group which may be cleaved upon exposure to an electromagnetic radiation source having a wavelength of between about 700 nm to about 1000 nm. Suitable near-infrared photocleavable groups include cyanine groups, including C4-dialkylamine-substituted heptamethine cyanines.

In these embodiments, the control system may command a source of electromagnetic radiation to irradiate the sample loading chamber and/or the bead-bound molecules provided therein with electromagnetic radiation for a pre-determined amount of time, at a pre-determined wavelength, and at a pre-determined intensity.

Following the release of the bead-bound molecules, the released molecules are then collected (step 513). In some embodiments, the released molecules are collected by transferring the released molecules through a channel (such as a branched channel) to another chamber, e.g., a sample collection chamber. This may be accomplished by establishing an electric field between the sample loading chamber and the other chamber of the separation conduit, e.g., a sample collection chamber. For instance, the released molecules are encouraged to move from the sample loading chamber to the sample collection chamber by applying an electric field between the sample loading chamber and the sample collection chamber. In some embodiments, the control system directs a switch to “open” (i.e., opening an electric current pathway) allowing current to flow between a first electrode in communication with the sample loading chamber and a second electrode in communication with the sample collection chamber. For instance, a current established between a sample loading chamber and a sample collection chamber may range from between about 10 mA to about 10 A. In other embodiments, the current established ranges from between 10 mA and 5 A. In other embodiments, the current established ranges from between 10 mA and 1 A. In other embodiments, the current established ranges from between 10 mA and 500 mA. In other embodiments, the current established ranges from between 10 mA and 100 mA. In other embodiments, a voltage established between a sample loading chamber and a sample collection chamber may range from between about 10V to about 10 kV. In other embodiments, a voltage established between a sample loading chamber and a sample collection chamber may range from between about 10V to about 5 kV. In other embodiments, a voltage established between a sample loading chamber and a sample collection chamber may range from between about 10V to about 1 kV. In other embodiments, a voltage established between a sample loading chamber and a sample collection chamber may range from between about 10V to about 500V. In other embodiments, a voltage established between a sample loading chamber and a sample collection chamber may range from between about 10V to about 250V. In other embodiments, a voltage established between a sample loading chamber and a sample collection chamber may range from between about 10V to about 100V.

In some embodiments, the collected released molecules may then be further transferred from the sample collection chamber to another vessel in communication therewith. For example, the released molecules transferred to the sample collection chamber may be pumped via a conduit to another vessel.

In other embodiments, the collected released molecules may be used in a downstream process, e.g., PCR, dPCR, sequencing, flow cytometry, etc. The released target molecules may then be used in one or more downstream processes, e.g., sequencing, amplification, further coupling, etc. In some embodiments, sequencing may be performed according to any method known to those of ordinary skill in the art. In some embodiments, sequencing methods include Sanger sequencing and dye-terminator sequencing, as well as next-generation sequencing technologies such as pyrosequencing, nanopore sequencing, micropore-based sequencing, nanoball sequencing, MPSS, SOLiD, Illumina, Ion Torrent, Starlite, SMRT, tSMS, sequencing by synthesis, sequencing by ligation, mass spectrometry sequencing, polymerase sequencing, RNA polymerase (RNAP) sequencing, microscopy-based sequencing, microfluidic Sanger sequencing, microscopy-based sequencing, RNAP sequencing, etc. Instruments and methods of sequencing are disclosed, for example, in PCT Publication Nos. WO2014144478, WO2015058093, WO2014106076 and WO2013068528, the disclosures of which are hereby incorporated by reference in their entireties.

Target Enrichment Using an Electrophoretic Device

The present disclosure also relates to a method of reducing the complexity of a nucleic acid sample by enriching for specific nucleic acid target sequences in the nucleic acid sample. In some embodiments, the present disclosure is directed to methods of enriching for specific target sequences in a nucleic acid sample using libraries of oligonucleotide probes. The nucleic acid sample enriched for the specific target sequences may then be used in downstream sequencing operations. In some embodiments, the methods are carried out in a closed system which mitigates risk of cross-contamination and/or or sample loss. In some embodiments, the methods described herein require no pipetting steps and thus prevent or mitigate bead loss, sample loss, and/or contamination.

In some embodiments, the present disclosure is directed to methods of target enrichment using any one of the electrophoretic devices described herein. The present disclosure is also directed to methods of sequencing using a target enriched sample, such as a target enriched sample prepared using any one of the electrophoretic devices described herein. In some embodiments, targeted sequencing, in general, enables the detection of known and novel variants in selected sets of genes or genomic regions. In some embodiments, the target enriched sample is sequenced using next-generation sequencing. For example, when a sample solution including nucleic acid sequences of interest are introduced to a sample loading chamber of a separation conduit pre-loaded with a plurality of functionalized beads, nucleic acids of interest are bound onto the bead surface through various chemistries. In some embodiments, unbound non-target nucleic acids and impurities may then be removed while the nucleic acids of interest remain bound to the functionalized beads. In some embodiments, nucleic acids of interest are released from the beads, and the released nucleic acids are then collected and sequenced.

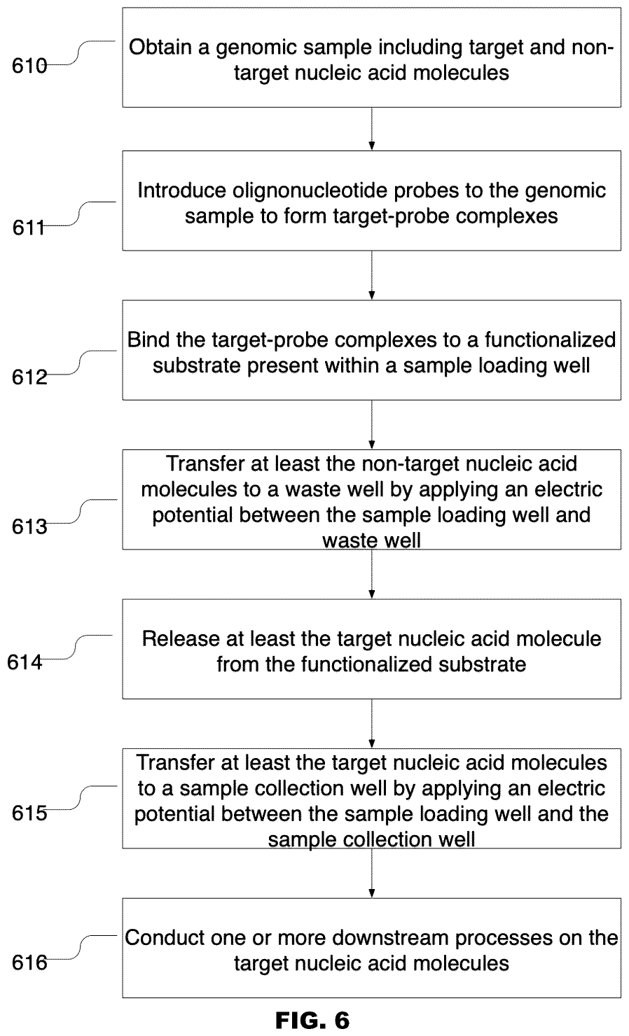

In some embodiments, and with reference to FIG. 6, target enrichment includes obtaining a genomic sample (step 610). In some embodiments, the obtained genomic sample is a sample derived from a mammalian subject, e.g., a human subject. In some embodiments, the obtained genomic sample is a blood sample, or a blood plasma sample obtained from a mammalian subject, e.g., a blood sample or a blood plasma sample obtained from a human subject. In some embodiments, the obtained genomic sample is in the form of cell-free nucleic acids. In some embodiments, the obtained genomic sample in the form of cell-free nucleic acids comprises DNA and/or RNA. In some embodiments, the cell-free DNA typically ranges in size from between about 200 bp to about 130 bp. In some embodiments, the cell-free DNA typically ranges in size from between about 190 bp to about 140 bp. In some embodiments, the cell-free DNA typically ranges in size from between about 180 bp to about 150 bp. Non-limiting examples of cell-free nucleic acids include circulating tumor DNA (ctDNA) and fetal cell-free DNA present in maternal blood and blood plasma. In some embodiments, the present disclosure also encompasses isolation of various types of cell-free RNA.

Alternatively, and in other embodiments, target enrichment includes obtaining a genomic sample, e.g., a genomic DNA sample acquired from a human patient. In some embodiments, the obtained genomic sample is sheared into fragments to provide a population of nucleic acid fragments. In some embodiments, shearing of the obtained genomic sample is effectuated using mechanical (e.g., nebulization or sonication) and/or enzymatic fragmentation (e.g., restriction endonucleases).

In some embodiments, the generated nucleic acid fragments are randomly sized. In some embodiments, the generated nucleic acid fragments have a length which are less than about 1000 base pairs. In other embodiments, the generated nucleic acid fragments comprises sequence fragments having a sequence size ranging from between about 100 to about 1000 base pairs in length. In yet other embodiments, the generated nucleic acid fragments comprises sequence fragments having a sequence size ranging from between about 500 to about 750 base pairs in length. In some embodiments, adapters, such as those including a specific barcode sequence, are then added via a ligation reaction to the population of nucleic acid.

Following the obtaining of the genomic sample (and/or the optional fragmentation of the obtained genomic sample), in some embodiments a pool of oligonucleotide probes, such as oligonucleotide probes conjugated to a first member of a pair of specific binding entities, are introduced to the obtained genomic sample or the population of nucleic acid fragments. In some embodiments, the pool of oligonucleotide probes are introduced to a buffer solution including the obtained genomic sample or the population of nucleic acid fragments. In some embodiments, the oligonucleotide probes are reference populations of nucleic acid sequences capable of hybridizing to complementary nucleic acid sequences within the genomic sample or the population nucleic acid fragments. In some embodiments, the oligonucleotide probes are designed to target desired genes, exons, and/or other genomic regions of interest within the genomic sample or the population of nucleic acid fragments. In some embodiments, the oligonucleotide probes are selected such that the oligonucleotide probes relate to, by way of non-limiting examples, a set of genes of interest, all of the exons of a genome, particular genetic regions of interest, disease or physiological states and the like.

In some embodiments, the oligonucleotide probes are DNA capture probes. In some embodiments, the DNA capture probes include a pool of Roche SeqCap EZ Probes (available from Roche Sequencing and Life Sciences, Indianapolis, IND). In some embodiments, a pool of Roche SeqCap EZ Probes include a mixture of different biotinylated single-stranded DNA oligonucleotides in solution, each with a specific sequence, where the length of individual oligonucleotides can range from about 50 nucleotides to about 100 nucleotides with a typical size of about 75 nucleotides. In some embodiments, a Roche SeqCap EZ Probe Pool can be used in sequence capture experiments to hybridize to targeted complementary fragments of a DNA sequencing library and thus to capture and enrich them relative to untargeted fragments of the same DNA sequencing library prior to sequencing. The DNA sequencing library may be constructed from genomic DNA for genome analysis, or from cDNA prepared from RNA or mRNA for transcriptome analysis, and it may be constructed from the DNA or cDNA of any species of organism from which these nucleic acids can be extracted.

In some embodiments, the oligonucleotide probes hybridize to a first subset of complementary nucleic acids within the genomic sample or nucleic acid fragments within the population of nucleic acid fragments which include the desired genes, exons, and/or other genomic regions of interest to form target-probe complexes having a first member of a pair of specific binding entities. In some embodiments, a second subset of nucleic acids or nucleic acid fragments within the obtained genomic sample or the solution of nucleic acid fragments, respectively, that do not include the desired genes, exons, and/or other genomic regions of interest do not form target-probe complexes and are referred to as “off-target nucleic acids” or “off-target fragments.” As such, following the introduction of the oligonucleotide probes, any solution for enrichment may include formed target-probe complexes, off-target nucleic acids or off-target fragments, and/or free probes (assuming that an excess amount of oligonucleotide probes are provided to any solution including adapter-ligated DNA fragments). In some embodiments, the solution for enrichment is provided in a buffer solution.

In some embodiments, the oligonucleotide probes are introduced to the genomic sample pre-loaded within a sample loading chamber of a separation conduit of an electrophoretic device, such as any of the electrophoretic devices described herein. In these embodiments, the solution for enrichment is formed within the sample loading chamber. Subsequently, a plurality of beads, e.g., a plurality of functionalized beads, may be introduced to the solution for enrichment present within the sample loading chamber.

In other embodiments, the oligonucleotide probes are reacted with the genomic sample in an upstream process, and then the resulting solution for enrichment is itself transferred to the sample loading chamber of a separation conduit of an electrophoretic device.

In some embodiments, the solution for enrichment, including the formed target-probe complexes, off-target nucleic acids and/or off-target fragments, is introduced to a sample loading chamber of a separation conduit of an electrophoretic device. In some embodiments, the sample loading chamber is pre-loaded with a plurality of beads, e.g., a plurality of functionalized beads. In some embodiments, any of the electrophoretic devices described herein may be utilized for target enrichment. In some embodiments, the electrophoretic device includes a separation conduit having no moving parts, e.g., no moving mechanical parts.

In some embodiments, the plurality of beads pre-loaded into the sample loading chamber are non-magnetic. In other embodiments, the plurality of beads pre-loaded into the sample loading chamber are magnetic. In some embodiments, each bead of the plurality of beads are functionalized with a plurality of second members of the pair of specific binding entities (e.g., avidin or streptavidin). In some embodiments, the sample loading chamber may be pre-loaded with between about 10 to about 10,000 functionalized beads. In other embodiments, the sample loading chamber may be pre-loaded with between about 10 to about 1000 functionalized beads. In yet other embodiments, the sample loading chamber may be pre-loaded with between about 10 to about 150 functionalized beads.

Regardless of whether the solution for enrichment is formed within the sample loading chamber or introduced to a sample loading chamber, the first members of the pair of specific binding entities of the target-probe complexes react with the second members of the pair of specific binding entities of the functionalized beads such that the target-probe complexes within the solution for enrichment become bound to the beads within the sample loading chamber of the separation conduit (step 612). Likewise, in some embodiments the first members of the pair of specific binding entities of any free probes in the solution for enrichment become bound to the functionalized beads. In this manner, the target-probe complexes and/or free probes become bound to the functionalities beads within the chamber. As such, the beads within the chamber include immobilized (i.e., bead bound) target-probe complex and free probes. Also included within the reaction chamber, in some embodiments, are unbound, off-target nucleic acids or off-target fragments.

In some embodiments, the target-probe complexes are allowed time to incubate with the functionalized beads. In some embodiments, an incubation period may range from between about 1 minute to about 120 minutes. In some embodiments, an incubation period may range from between about 1 minute to about 60 minutes. In other embodiments, an incubation period may range from between about 1 minute to about 40 minutes. In yet other embodiments, an incubation period may range from between about 1 minute to about 20 minutes.

Following the binding of the target-probe complexes to the functionalized beads and/or the binding of free-probes to the beads, unbound off-target nucleic acids, off-target fragments, reagents, and/or impurities are then removed from the sample loading chamber of the separation conduit (step 613). In some embodiments, removal of the off-target fragments that were not complementary to any of the oligonucleotide probes introduced to the solution for enrichment enriches the remaining immobilized target genomic material.

In some embodiments, the unbound non-functionalized molecules and/or impurities are transported from the sample loading chamber, through a branched channel, and to another chamber, e.g., a waste collection chamber. This may be accomplished by establishing an electric field between the sample loading chamber and the other chamber of the separation conduit, e.g., a waste collection chamber.

In some embodiments, the chambers and/or channels of the separation conduits of the electrophoretic devices utilized are pre-loaded with one or more gels and/or electrolytic compositions. For instance, in some embodiments, one or more channels in communication with a sample loading chamber and/or other chambers of the separation conduit, e.g., waste collection chamber and/or sample loading chamber, are pre-loaded with a gel. In some embodiments, the gel is agarose and/or PAGE. For instance, a gel may be infused into the one or more channels (or a branched channel having one or more levels of branching) prior to or subsequent to the introduction of the sample into the sample loading chamber. In some embodiments, the gel is pumped into the one or more channels (or branched channel) from a reservoir and through one or more conduits in fluidic communication with the one or more channels (or the various branches of the branched channel). In some embodiments, a gel is formed in situ within the one or more channels (or the branched channel). In some embodiments, the gel serves as a sieving matrix and/or a filter to retain the plurality of beads within the sample loading chamber.

In some embodiments, chambers of the separation conduit of the electrophoretic device are also pre-loaded with one or more electrolytes. In some embodiments, one or more collection chambers of the separation conduit are pre-loaded with between about 0.2 mL to about 1000 mL of the one or more electrolytes. In other embodiments, one or more collection chambers of the separation conduit are pre-loaded within between about 1 mL to about 500 mL of the one or more electrolytes. In some embodiments, the one or more electrolytes are each leading electrolytes. In some embodiments, the one or more electrolytes are selected from are selected from HCl-Histidine, Tris/Acetate/EDTA (TAE), and Tris/Borate/EDTA (TBE). In some embodiments, each of a waste collection chamber and a sample collection chamber are pre-loaded within one or more electrolytes, e.g., one or more leading electrolytes. In some embodiments, the one or more electrolytes introduced to the waste collection chamber as the same as those introduced to the sample collection chamber. In other embodiments, the one or more electrolytes introduced to the waste collection chamber are different than those introduced to the sample collection chamber.

In some embodiments, the sample loading chamber is also pre-loaded with one or more electrolytes. Alternatively, the one or more electrolytes may be introduced to the sample loading chamber following the introduction of the sample to be purified. In some embodiments, the sample loading chamber is pre-loaded within between about 1 μL to about 100 μL of the one or more electrolytes. In other embodiments, the sample loading chamber is pre-loaded within between about 0.01 mL to about 10 mL of the one or more electrolytes. In some embodiments, the one or more electrolytes are each trailing electrolytes. In some embodiments, the one or more electrolytes are selected from TAPS-TRIS, Tris/Acetate/EDTA (TAE), and Tris/Borate/EDTA (TBE). In some embodiments, the plurality of beads and the one or more electrolytes are introduced as a mixture to the sample loading chamber. In some embodiments, the electrolyte is pre-loaded prior to sample/bead loading. In other embodiments, the electrolyte is pre-mixed with the sample/bead prior to loading into the device.

In some embodiments, the unbound off-target nucleic acids, off-target fragments, reagents, and/or impurities are encouraged to move from the sample loading chamber to the waste collection chamber by applying an electric field between the sample loading chamber and the waste collection chamber. In some embodiments, the control system directs a switch to “open” (i.e., opening an electric current pathway) allowing current to flow between a first electrode in communication with the sample loading chamber and a second electrode in communication with the waste collection chamber. For instance, a current established between a sample loading chamber and a waste collection chamber may range from between about 10 mA to about 10 A. In other embodiments, the current established ranges from between 10 mA and 5 A. In other embodiments, the current established ranges from between 10 mA and 1 A. In other embodiments, the current established ranges from between 10 mA and 500 mA. In other embodiments, the current established ranges from between 10 mA and 100 mA. In other embodiments, a voltage established between a sample loading chamber and a waste collection chamber may range from between about 10V to about 10 kV. In other embodiments, a voltage established between a sample loading chamber and a waste collection chamber may range from between about 10V to about 5 kV. In other embodiments, a voltage established between a sample loading chamber and a waste collection chamber may range from between about 10V to about 1 kV. In other embodiments, a voltage established between a sample loading chamber and a waste collection chamber may range from between about 10V to about 500V. In other embodiments, a voltage established between a sample loading chamber and a waste collection chamber may range from between about 10V to about 250V. In other embodiments, a voltage established between a sample loading chamber and a waste collection chamber may range from between about 10V to about 100V.

Following the removal of substantially all off-target nucleic acids, off-target fragments, reagents, and/or impurities from the chamber of the separation conduit, the target molecules or target molecule complexes are removed from the sample loading chamber. In some embodiments, the target molecules are released from the beads (step 614) and subsequently collected (step 615). In some embodiments, the target molecules or target molecule complexes are released by introducing a chemical or an enzyme to the sample loading chamber. In other embodiments, the target molecules or target molecule complexes are released by irradiating the sample loading chamber with electromagnetic radiation or by heating the sample loading chamber.

In other embodiments, a reagent (e.g., an enzyme) is introduced to effectuate release. Examples of suitable enzymes include trypsin (which cleaves the peptide bonds at the carboxyl end of lysine and arginine residues) and clostripain (which cleaves at the carboxyl side of arginine residues). In some embodiments, the reagent is allowed time to incubate with the bead-bound target-probe complexes and/or the bead-bound free probes. In some embodiments, an incubation period may range from between about 1 minute to about 60 minutes. In other embodiments, an incubation period may range from between about 1 minute to about 40 minutes. In yet other embodiments, an incubation period may range from between about 1 minute to about 20 minutes.

In some embodiments, the control system of the electrophoretic device may command one or more thermal regulation modules to heat the sample loading chamber and the target molecules or target molecule complexes therein to a temperature ranging from between about 30° C. to about 95° C. In other embodiments, the control system may command one or more thermal regulation modules to heat the sample loading chamber and the target molecules or target molecule complexes therein to a temperature ranging from between about 40° C. to about 70° C. In yet other embodiments, the control system may command one or more thermal regulation modules to heat the sample loading chamber and the target molecules or target molecule complexes therein to a temperature ranging from between about 45° C. to about 65° C. In some embodiments, the control system commands one or more thermal regulation modules in communication with the sample loading chamber to heat the sample loading chamber and/or the target molecules or target molecule complexes to temperature in which two hybridized strands of a nucleic acid molecule become denatured, e.g., 90° C. to about 95° C.

Following the release of the target molecules or target molecule complexes, the target molecules or target molecule complexes are then collected. In some embodiments, the released molecules are collected by transferring the released molecules through a channel (such as a branched channel) to another chamber, e.g., a sample collection chamber (step 615). This may be accomplished by establishing an electric field between the sample loading chamber and the other chamber of the separation conduit, e.g., a sample collection chamber. For instance, the target molecules or target molecule complexes are encouraged to move from the sample loading chamber to the sample collection chamber by applying an electric field between the sample loading chamber and the sample collection chamber. In some embodiments, the control system directs a switch to “open” (i.e., opening an electric current pathway) allowing current to flow between a first electrode in communication with the sample loading chamber and a second electrode in communication with the sample collection chamber. For instance, a current established between a sample loading chamber and a sample collection chamber may range from between about 10 mA to about 10 A. In other embodiments, the current established ranges from between 10 mA and 5 A. In other embodiments, the current established ranges from between 10 mA and 1 A. In other embodiments, the current established ranges from between 10 mA and 500 mA. In other embodiments, the current established ranges from between 10 mA and 100 mA. In other embodiments, a current established between a sample loading chamber and a sample collection chamber may range from between about 10V to about 10 kV. In other embodiments, a current established between a sample loading chamber and a sample collection chamber may range from between about 10V to about 5 kV. In other embodiments, a current established between a sample loading chamber and a sample collection chamber may range from between about 10V to about 1 kV. In other embodiments, a current established between a sample loading chamber and a sample collection chamber may range from between about 10V to about 500V. In other embodiments, a current established between a sample loading chamber and a sample collection chamber may range from between about 10V to about 250V. In other embodiments, a current established between a sample loading chamber and a sample collection chamber may range from between about 10V to about 100V.

In some embodiments, the collected target molecules or target molecule complexes molecules may then be further transferred from the sample collection chamber to another vessel in communication therewith. For example, the target molecules or target molecule complexes transferred to the sample collection chamber may be pumped via a conduit to another vessel.

In other embodiments, the collected target molecules or target molecule complexes may be used in a downstream process, e.g., PCR, dPCR, ddPCR, sequencing, flow cytometry, etc. In some embodiments, sequencing may be performed according to any method known to those of ordinary skill in the art.

Reactions/Solid-State Synthesis Carried Out within a Separation Conduit of an Electrophoretic Device

The present disclosure provides, in some embodiments, a method of performing one or more solid-phase reactions in a separation conduit of an electrophoretic device. In general, the methods of performing one or more solid-phase reactions in a separation conduit of a electrophoretic device of the present disclosure comprise (i) binding a subset of appropriately functionalized molecules within an input sample to functionalized beads present within a sample loading chamber of a separation conduit; (ii) transferring unbound molecules and/or impurities to a waste collection chamber; (iii) releasing the bound molecules from the beads; and (iv) transferring the released molecules to a sample collection chamber. In some embodiments, one or more reagents may be optionally introduced into the sample loading chamber of the separation conduit to derivatize the subset of molecules bound to the functionalized beads. In other embodiments, the released molecules are transferred to an intermediate chamber where they may be reacted with one or more reagents. The product of that reaction may then be transferred to a sample collection chamber.

For example, the molecules bound to the beads may be an oligonucleotide and the reagents may include nucleotides or short oligonucleotides for conjugation. By way of another example, the molecules bound to the beads may be peptides and the reagents may include amino acids or short peptides for conjugation. In some embodiments, the molecules bound to the beads may be DNA or RNA aptamers; and the reagents may include small molecules, peptides, proteins or cells which specifically bind to surface-immobilized aptamer molecules. One or more different reactions may take place sequentially.

After all desired reactions have been carried out, the subset of molecules bound to the beads are then released from the beads and subsequently collected as described herein. In some embodiments, the released subset of molecules may then be used in one or more downstream processes.

### EXAMPLES

### Example 1—Device Structure and Loading

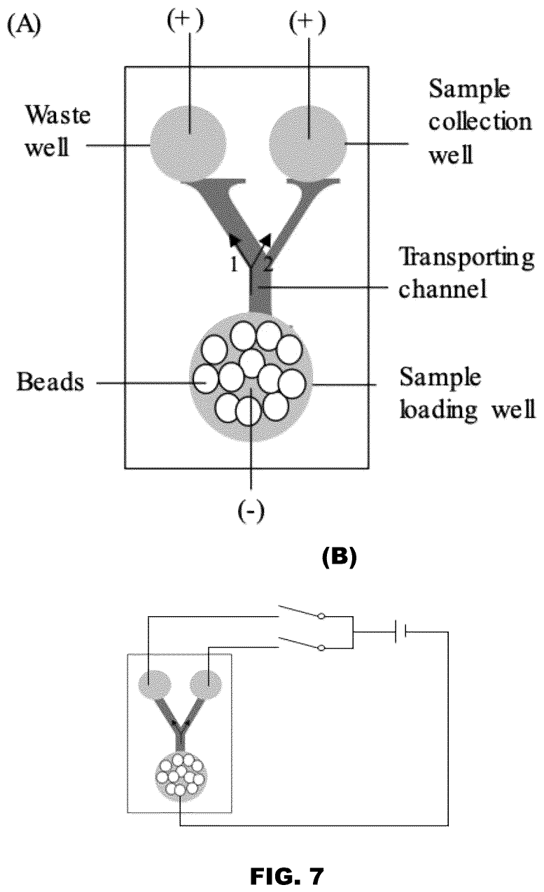

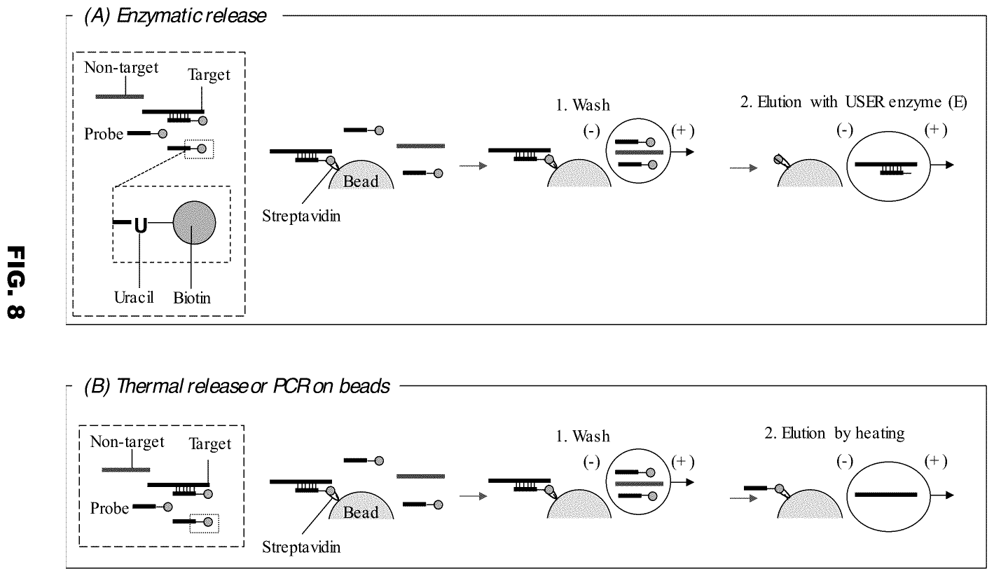

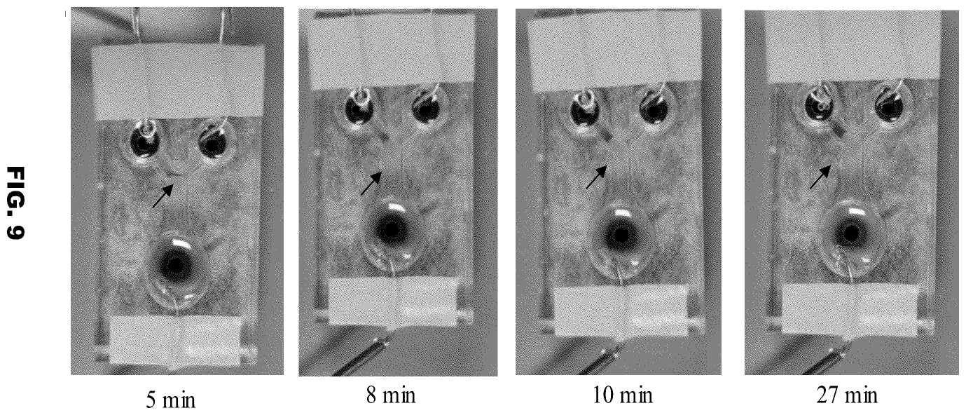

FIG. 7 depicts an electrophoretic device including three different chambers for sample loading, waste and sample collection, respectively, interconnected by a branched channel. In some embodiments, the channel may be filled with a gel (e.g., agarose, PAGE) which can serve as a sieving matrix and/or a filter to retain beads within the sample loading chamber. Electrolytes, i.e., electrically-conducting media, may be loaded into the waste and sample collection chambers, while capture beads (e.g., streptavidin-coated magnetic or non-magnetic polymeric beads) suspended in an electrolyte are loaded into the sample loading chamber. A sample mixture including both target and non-target molecules/complexes may then loaded into the sample loading chamber followed by its incubation with the beads at a constant temperature. During the incubation, molecules/complexes of interest may be bound to the bead surface through covalent (e.g., carboxyl-amine) or affinity binding (e.g., streptavidin-biotin). After the incubation, an electric current may be applied between the sample loading chamber and the waste collection chamber, during which unbound non-target molecules and other charged impurities are electrophoretically moved through the gel and into the waste collection chamber. Subsequently, the electric field is turned off for the next elution step in which target molecules/complexes may be released from the bead surface, which may occur either enzymatically or thermally. The electric current pathway may be then subsequently switched to between the sample collection chamber and sample loading chamber by which released target molecules/complexes are electro-migrated into the sample collection chamber.

### Example 2—Device Structure and Transport of Molecules

The electrophoretic device may be fabricated by laminating multiple layers of laser-cut plastic sheets (e.g., PMMA) using double-sided adhesive. A first prototype electrophoretic device was tested to demonstrate its functionality for molecular transportation using isotachophoresis. A branched transfer channel was first filled with 1% agarose gel prepared with 20 mM of leading electrolyte (LE) buffer (20 mM HCl-Histidine). Both the waste and sample collection chambers were then filled with 10o mL of LE (100 mM HCl-Histidine). 50 mM of trailing electrolyte (TE) buffer (50 mM TAPS-TRIS) spiked with Brilliant Blue color dye (whose electrophoretic mobility is similar to DNAs) was then loaded into the sample loading chamber. When the electric current was applied between the sample loading chamber (FIG. 9) and the waste collection chamber, a blue band indicated that dye molecules were formed after 5 min near the channel bifurcation region and started migrating towards the waste collection chamber. The band was observed to be darker and thicker over time, representing the focusing effect of isotachophoresis.

### Example 3—Avenio ctDNA Target Enrichment

In the Avenio ctDNA (circulating tumor DNA) analysis workflow, target DNA molecules of interest in a library were hybridized with a pool of biotinylated probes in the capture panel. When this hybridized sample was loaded into the sample loading chamber and incubated with the streptavidin-coated beads, target-probe complexes and free probes became bound to the bead surface via streptavidin-biotin binding. Off-target molecules were then electrophoretically transported from a sample loading chamber to a waste collection chamber by electric field application between those two chambers. After the washing step, either temperature-induced melting or enzymatic cleavage was used to release target molecules or target-probe complexes from the bead surface. For the thermal release, the temperature of the sample loading chamber was raised to 95° C. to denature the target strands from the probe attached to the bead surface. For the specific enzymatic cleavage, uracil was placed between probe sequence and biotin. After the washing, the beads were incubated with USER enzyme to cleave the uracil site and release the target-probe complexes from the bead surface. Released target molecules or target-probe complexes were then transported into the sample collection chamber by applying an electric current between sample loading and collection chambers. The eluate in the sample collection chamber was finally retrieved by pipetting and transferred for subsequent processing, e.g., sequencing.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications, and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1. An electrophoretic device comprising: one or more independently operable separation conduits; wherein each of the one or more independently operable separation conduits comprise at least one sample loading chamber, at least one waste collection chamber, and at least one sample collection chamber, wherein the at least one sample loading, the at least one waste collection, and the at least one sample collection chambers are fluidically coupled to each other through a branched transfer channel; and wherein the at least one sample loading chamber is in communication with at least one first electrode, the at least one waste collection chamber is in communication with at least one second electrode, and the at least one sample collection chamber is in communication with at least one third electrode.

2. The electrophoretic device of claim 1, further comprising at least two electrical traces or wires, wherein a first of the at least two electrical traces or wires couple the at least one first electrode to the at least one second electrode.

3. (canceled)

4. The electrophoretic device of claim 2, wherein a second of the at least two electrical traces or wires couple the at least one first electrode to the at least one third electrode.

5. The electrophoretic device of claim 1, further comprising a control system.

6. The electrophoretic device of claim 1, further comprising one or more feedback control devices.

7. The electrophoretic device of claim 1, further comprising one or more heating and/or cooling modules.

8. The electrophoretic device of claim 1, wherein a first portion of a wall of each of the at least one sample loading chambers comprises a first aperture in communication with an inlet of the branched transfer channel.

9. The electrophoretic device of claim 8, wherein the at least one sample loading chamber includes a plurality of beads, and wherein the first aperture is smaller than an average diameter of the plurality of beads within the at least one sample loading chamber.

10. (canceled)

11. The electrophoretic device of claim 9, wherein a second portion of the wall comprises a ductal opening, and wherein the ductal opening is larger than the average diameter of the plurality of beads within the at least one sample loading chamber.

12. (canceled)

13. The electrophoretic device of claim 1, wherein the electrophoretic device comprises no mechanically moving parts.

14. The electrophoretic device of claim 1, wherein the at least one sample loading chamber comprises a volume ranging from between about 0.1 μL to about 5 mL.

15. The electrophoretic device of claim 14, wherein the volume of the at least one sample loading chamber ranges from between about 0.1 mL to about 1 mL.

16. The electrophoretic device of claim 1, further comprising two sample collection chambers, wherein a first of the two sample collection chambers is in communication with the branched transfer conduit, and wherein the first of the two sample collection chambers is in further communication with a second of the two sample collection chambers through an intermediate channel.

17. The electrophoretic device of claim 1, wherein the branched transfer channel is pre-loaded with a gel.

18. (canceled)

19. The electrophoretic device of claim 1, wherein the at least one waste collection chamber and the at least one sample collection chamber are each pre-loaded with one or more electrolytes wherein the one or more electrolytes are one or more leading electrolytes.

20. (canceled)

21. (canceled)

22. The electrophoretic device of claim 19, wherein the at least one sample loading chamber includes one or more trailing electrolytes.

23. (canceled)

24. (canceled)

25. (canceled)

26. (canceled)

27. A method of obtaining a population of target nucleic acid sequences for sequencing comprising: (a) introducing a pool of oligonucleotide probes to an obtained genomic sample to form target-probe complexes, wherein the pool of oligonucleotide probes comprises reference nucleic acid sequences capable of hybridizing to complementary target nucleic acid sequences within the obtained genomic sample, and wherein the oligonucleotide probes comprise a first member of a pair of specific binding entities; (b) introducing a solution including the formed target-probe complexes to a sample loading chamber of a separation conduit pre-loaded with a plurality of beads to form bead-bound target-probe complexes, wherein the plurality of beads are functionalized with a second member of the pair of specific binding entities; (c) transferring off-target nucleic acids to a waste collection chamber in communication with the sample loading chamber by establishing an electrical field between the sample loading chamber and the waste collection chamber; and (d) transferring the target nucleic acids to a sample collection chamber in communication with the sample loading chamber by establishing an electrical field between the sample loading chamber and the sample collection chamber.

28. The method of claim 27, further comprising releasing the target-probe complexes from the plurality of beads.

29. The method of claim 28, wherein the formed target-probe complexes comprise a cleavable moiety.

30. (canceled)

31. (canceled)

32. The method of claim 27, further comprising releasing the target nucleic acids from the target-probe complexes.

33-71. (canceled)
